(19) United States
(12) Patent Application Publication
(10) Pub. No.: US 2023/0103276 A9
(43) Pub. Date: Mar. 30, 2023

(54) IMPAIREMENT SCREENING SYSTEM AND METHOD

(71) Applicant: CannSight Technologies Inc., West Vancouver (CA)

(72) Inventors: Yaser Mohammadian Roshan, West Vancouver (CA); Ehsan Daneshi Kohan, Coquitlam (CA); Aaron North, Thornhill (CA); Ilan Nachim, Thornhill (CA)

(73) Assignee: CannSight Technologies Inc., West Vancouver (CA)

(21) Appl. No.: 16/892,683

(22) Filed: Jun. 4, 2020

Related U.S. Application Data
(60) Provisional application No. 62/858,307, filed on Jun. 6, 2019.

(51) Int. Cl. A61B 5/00; G06T 7/292; A61B 5/16; A61B 5/11; A61B 3/14; A61B 5/0205

(52) U.S. Cl. CPC A61B 5/4845; G06T 7/292; A61B 5/163; A61B 5/4023; A61B 5/1118; A61B 3/14; A61B 5/7445; A61B 5/6803; A61B 5/0205; G06T 2207/30041

(57) ABSTRACT

A system for screening impairment of a subject includes an imaging device and a display connected to a controller, the controller configured to send a first eye test signal to the imaging device and a second eye test signal to the display to initiate an eye test, and further configured to receive an eye test feedback signal based on captured images of subject eye movement during the eye test. The controller is configured to send a first cognitive test signal to the imaging device and a second cognitive test signal to the display to initiate a cognitive test, and is further configured to receive a cognitive test feedback signal based on captured images of subject eye movement during the cognitive test. A balance sensor is connected to the controller, the controller configured to receive a balance test feedback signal from the balance sensor indicative of subject movement during a balance test. A physiological sensor is connected to the controller, the controller configured to receive a physiological activity feedback signal from the physiological sensor indicative of subject physiological activity. The controller is configured to generate an impairment indication based on the eye test feedback signal, the cognitive test feedback signal, the balance test feedback signal, and the physiological activity feedback signal. A method for screening impairment of a subject is also disclosed.

### CROSS-REFERENCE TO RELATED APPLICATIONS

This application is related to and claims the benefit of priority of U.S. provisional application No. 62/858,307 filed on Jun. 6, 2019 incorporated herein by reference in its entirety.

Embodiments of the invention relate generally to screening for neurological impairment caused for example by the influence of drugs, alcohol, lack of sleep, or any other related neurological or cognitive disorder. More specifically, the present disclosure provides systems and methods for performing eye tests, cognitive tests (e.g. testing neurocognitive function), balance tests along with providing physiological feedback for screening and determining subject impairment.

### BACKGROUND OF THE INVENTION

Traffic accidents are predominantly caused by Driving Under Influence (DUI) or Driving While Impaired (DWI). For people in Europe between the age of 15 and 29, DUI is one of the main causes of mortality. According to the National Highway Traffic Safety Administration, DUI and alcohol-related crashes cause approximately $37 billion in damages annually. Accidents due to impairment are not limited to driving and also includes impairment at work places in certain sectors such as construction, transportation, manufacturing, oil and gas, etc. For the sake of generality, performance of an activity under impairment is referred to herein as Acting While Impaired (Awl).

AWI is not only limited to alcohol consumption, but it also includes the consumption of recreational drugs such as cannabis products such as marijuana or hashish, as well as prescription drugs such as opioids and benzodiazepines.

Drugs, including alcohol, have a profound effect upon human eye movement and eye reaction to light stimuli, human cognitive and neurologic behavior, and bio signals. Field sobriety tests being performed by authorities in AWI cases typically include the evaluation of eyes which generally includes tests of equal eye size, convergence, nystagmus, and smooth pursuit, and also cognitive tests including the one-leg stand test and the walk-and-run test. The main problem in performing these tests is the subjectivity of the results based on the observation and experience of the agent. This impacts the accuracy and validity of the test results. Also, there are potential inaccuracies when recording the results, and questions as to whether any of these deficiencies can be argued such that any evidence is inadmissible in court or related administration proceedings.

Thus, there is a need in the art for improved methods and systems of AWI screening that improve system accuracy, while also improving record keeping and test administration abilities during the screening process.

### SUMMARY OF THE INVENTION

In one embodiment, a system for screening impairment of a subject includes an imaging device and a display connected to a controller, the controller configured to send a first eye test signal to the imaging device and a second eye test signal to the display to initiate an eye test, and further configured to receive an eye test feedback signal based on captured images of subject eye movement during the eye test, and the controller configured to send a first cognitive test signal to the imaging device and a second cognitive test signal to the display to initiate a cognitive test, and further configured to receive a cognitive test feedback signal based on captured images of subject eye movement during the cognitive test; a balance sensor connected to the controller, the controller configured to receive a balance test feedback signal from the balance sensor indicative of subject movement during a balance test; and a physiological sensor connected to the controller, the controller configured to receive a physiological activity feedback signal from the physiological sensor indicative of subject physiological activity; wherein the controller is configured to generate an impairment indication based on the eye test feedback signal, the cognitive test feedback signal, the balance test feedback signal, and the physiological activity feedback signal. In one embodiment, the controller is configured to send an impairment determination signal to the display based on the impairment indication. In one embodiment, the imaging device includes a first camera. In one embodiment, the imaging device includes a first and second camera. In one embodiment, the display includes a plurality of light elements. In one embodiment, the plurality of light elements are a plurality of LED elements. In one embodiment, an optical diffuser ii s configured to cover the plurality of light elements. In one embodiment, the plurality of light elements includes a linear array of light elements. In one embodiment, the plurality of light elements includes a plurality of linear arrays of light elements. In one embodiment, a first array of the plurality of linear arrays of light elements is disposed horizontally. In one embodiment, a second array of the plurality of linear arrays of light elements is disposed vertically. In one embodiment, the plurality of light elements includes a plurality of linear arrays of light elements disposed parallel to each other. In one embodiment, the plurality of light elements includes a plurality of linear arrays of light elements disposed perpendicular to each other. In one embodiment, the plurality of light elements includes a plurality of linear arrays of light elements disposed in a symmetrical grid pattern. In one embodiment, the grid pattern includes a higher density of lights in central portions of the grid and a lower density of lights in peripheral portions of the grid. In one embodiment, the display includes a display screen. In one embodiment, the goggles comprising the system, and the display is configured within a viewing cavity. In one embodiment, an administrator display configured on an external surface of the goggles. In one embodiment, an administrator display configured out of the subject's view during testing. In one embodiment, the system includes an imaging illumination element. In one embodiment, the illumination element is an infrared light element. In one embodiment, the balance sensor is an accelerometer, gyroscope, magnetometer, shoe or insole force sensor, or wearable activity monitoring sensor. In one embodiment, the imaging device functions as the balance sensor. In one embodiment, the physiological sensor is a heart rate sensor, a blood pressure sensor, a body tremor sensor, an oral moisture sensor, an electrodermal activity monitor, a body temperature sensor, sweat and skin conductance sensor, a muscle tone sensor, a frequency response sensor, an electromyography sensor, a glucometer, a blood analyzer, a stethoscope, a dermatoscope, an otoscope, an ophtalmoscope, an endoscope or an ultrasound scanner. In one embodiment, one or more sensors is disposed on a wristband. In one embodiment, the eye test includes at least one of a resting nystagmus eye test, a horizontal gaze nystagmus eye test, a vertical gaze nystagmus eye test, a lack of smooth pursuit eye test, an equal pupil eye test, a nystagmus at maximum deviation eye test, a nystagmus prior to 45 degrees eye test, a non-convergence eye test, a pupil rebound dilation test, a Hippus test, a red-eye (bloodshot) test, a watery eye test, and an eyelid twitching test. In one embodiment, the controller is electrically coupled to the imaging module, display module, balance sensor and physiological sensor. In one embodiment, the controller is wirelessly connected to at least one of the imaging modules, display module, balance sensor and physiological sensor. In one embodiment, the controller is configured to generate the impairment determination signal based on at least one of image analysis, data analysis, data visualization, and data integration. In one embodiment, the system includes a hand controller configured to measure a reaction time to a light signal. In one embodiment, the system includes a hand controller configured to measure a reaction time to an auditory signal. In one embodiment, the system is a portable system further comprising a battery for powering the controller imaging module, display module, balance sensor and physiological sensor. In one embodiment, the system is a handheld system further comprising a battery for powering the controller imaging module, display module, balance sensor and physiological sensor. In one embodiment, the system is a goggle system further comprising a battery for powering the controller imaging module, display module, balance sensor and physiological sensor. In one embodiment, the goggle system includes a flexible surface configured to contour against a subject's face. In one embodiment, the goggle system includes a head-mounting mechanism for attachment to the subject's head. In one embodiment, the head-mounting mechanism is an adjustable or elastic band. In one embodiment, the system includes detachable components, and two or more of the controllers, imaging module, display module, balance sensor and physiological sensor are detachable from the system. In one embodiment, mobile device detachable from the system includes two or more of the controllers, imaging module, display module, balance sensor and physiological sensor.

In one embodiment, a method for screening impairment of a subject includes the steps of receiving an eye test feedback signal from an imaging device based on captured images of subject eye movement during an eye test, receiving a cognitive test feedback signal from the imaging device based on captured images of subject eye movement during a cognitive test, receiving a balance test feedback signal from a balance sensor indicative of subject movement during a balance test, receiving a physiological activity feedback signal from a physiological sensor indicative of subject physiological activity, and generating an impairment indication based on the eye test feedback signal, the cognitive test feedback signal, the balance test feedback signal, and the physiological activity feedback signal. In one embodiment, the method includes the step of generating an impairment determination signal based on the impairment indication. In one embodiment, the method includes the step of measuring the physiological activity indicated in the physiological activity feedback signal from a physiological sensor contacting the subject's skin.

In one embodiment, a system for screening impairment of a subject includes an imaging device and a display connected to a controller, the controller configured to send a first eye test signal to the imaging device and a second eye test signal to the display to initiate an eye test, and further configured to receive an eye test feedback signal based on captured images of subject eye movement during the eye test, and the controller configured to send a first cognitive test signal to the imaging device and a second cognitive test signal to the display to initiate a cognitive test, and further configured to receive a cognitive test feedback signal based on captured images of subject eye movement during the cognitive test; where the controller is configured to generate an impairment indication based on the eye test feedback signal and the cognitive test feedback signal. In one embodiment the system includes a balance sensor connected to the controller, the controller configured to receive a balance test feedback signal from the balance sensor indicative of subject movement during a balance test; where the controller is configured to generate the impairment indication based on the balance test feedback signal. In one embodiment the system includes a physiological sensor connected to the controller, the controller configured to receive a physiological activity feedback signal from the physiological sensor indicative of subject physiological activity; where the controller is configured to generate the impairment indication based on the physiological activity feedback signal.

In one embodiment, a system for screening impairment of a subject includes a balance sensor connected to a controller, the controller configured to receive a balance test feedback signal from the balance sensor indicative of subject movement during a balance test; where the controller is configured to generate an impairment indication based on the balance test feedback signal. In one embodiment, the system includes a physiological sensor connected to the controller, the controller configured to receive a physiological activity feedback signal from the physiological sensor indicative of subject physiological activity; wherein the controller is configured to generate the impairment indication based on the physiological activity feedback signal. In one embodiment the system includes an imaging device and a display connected to the controller, the controller configured to send a first eye test signal to the imaging device and a second eye test signal to the display to initiate an eye test, and further configured to receive an eye test feedback signal based on captured images of subject eye movement during the eye test, and the controller configured to send a first cognitive test signal to the imaging device and a second cognitive test signal to the display to initiate a cognitive test, and further configured to receive a cognitive test feedback signal based on captured images of subject eye movement during the cognitive test; where the controller is configured to generate the impairment indication based on the eye test feedback signal and the cognitive test feedback signal.

In one embodiment, a system for screening impairment of a subject includes a physiological sensor connected to a controller, the controller configured to receive a physiological activity feedback signal from the physiological sensor indicative of subject physiological activity; where the controller is configured to generate an impairment indication based on the physiological activity feedback signal. In one embodiment, the system includes a balance sensor connected to the controller, the controller configured to receive a balance test feedback signal from the balance sensor indicative of subject movement during a balance test; where the controller is configured to generate the impairment indication based on the balance test feedback signal.

In one embodiment, the system includes an imaging device and a display connected to the controller, the controller configured to send a first eye test signal to the imaging device and a second eye test signal to the display to initiate an eye test, and further configured to receive an eye test feedback signal based on captured images of subject eye movement during the eye test, and the controller configured to send a first cognitive test signal to the imaging device and a second cognitive test signal to the display to initiate a cognitive test, and further configured to receive a cognitive test feedback signal based on captured images of subject eye movement during the cognitive test; where the controller is configured to generate the impairment indication based on the eye test feedback signal and the cognitive test feedback signal.

### BRIEF DESCRIPTION OF THE DRAWINGS

The following figures set forth embodiments in which like reference numerals denote like parts. Embodiments are illustrated by way of example and not by way of limitation in the accompanying figures.

FIG. 1A is a system diagram of impairment screening system components according to one embodiment, and FIG. 1B is a schematic diagram of an impairment screening device according to one embodiment.

FIG. 2 shows the handheld device screen and a screenshot of the software running on it according to one embodiment.

FIG. 3 shows an alternative handheld device screen according to one embodiment.

FIG. 4 shows the software running on a monitor connected to the device (wired or wireless) according to one embodiment.

FIG. 5 shows the software running on a handheld tablet connected to the device (wired or wireless) according to one embodiment.

FIG. 6 shows the top view of the impairment screening device according to one embodiment according to one embodiment.

FIG. 7 shows the front view of the impairment screening device for the case of two separate eye spaces according to one embodiment.

FIG. 8A shows the front view of the impairment screening device for the case of one eye space according to one embodiment. FIG. 8B shows the front view of the impairment screening device for the case of one eye space including an optical diffuser according to one embodiment.

FIG. 9 demonstrates two of the different pattern examples of white LEDs on the LED-driver printed circuit board (depending on the two or one eye space system) and defined the naming convention for each white LED according to one embodiment.

FIG. 10 is a flowchart showing steps of Resting Nystagmus test according to one embodiment.

FIG. 11 is a flowchart showing steps of Horizontal Gaze Nystagmus test for the two separate eye space system according to one embodiment.

FIG. 12 is a flowchart showing steps of Horizontal Gaze Nystagmus test for the one eye space system according to one embodiment.

FIG. 13 is a flowchart showing steps of Vertica I Gaze Nystagmus test for the two separate eye space system according to one embodiment.

FIG. 14 is a flowchart showing steps of Vertica I Gaze Nystagmus test for the one eye space system according to one embodiment.

FIG. 15 is a flowchart showing steps of Equal Pupils test according to one embodiment.

FIG. 16 is a flowchart showing steps of Lack of Smooth Pursuit test for the two separate eye space system according to one embodiment.

FIG. 17 is a flowchart showing steps of Lack of Smooth Pursuit test for the one eye space system according to one embodiment.

FIG. 18 is a flowchart showing steps of Nystagmus at Maximum Deviation test for the two separate eye space system according to one embodiment.

FIG. 19 is a flowchart showing steps of Nystagmus at Maximum Deviation test for the one eye space system according to one embodiment.

FIG. 20 is a flowchart showing steps of Nystagmus Prior to 45 Degrees test for the two separate eye space system according to one embodiment.

FIG. 21 is a flowchart showing steps of Nystagmus Prior to 45 Degrees test for the one eye space system according to one embodiment.

FIG. 22 is a flowchart showing steps of Non-convergence test for the two separate eye space system according to one embodiment.

FIG. 23 is a flowchart showing steps of Non-convergence test for the one eye space system according to one embodiment.

FIG. 24 is the hand controller for the reaction tests according to one embodiment.

FIG. 25 shows the front view of the device in the design with one horizontal and one vertical row of white LEDs according to one embodiment.

FIG. 26 shows the complete testing station including the camera(s) according to one embodiment.

FIG. 27 shows a setup of a system according to one embodiment.

FIG. 28 shows a setup of a portable cart system according to one embodiment.

FIG. 29 shows a setup of a portable cart system with horizontal and vertical LED arrays according to one embodiment.

FIG. 30 shows setup of a briefcase system according to one embodiment

FIG. 31 is a flow chart of a method for screening impairment of a subject according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods of screening for impairment. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a comprehensive impairment screening system, device and method.

Advantageously, embodiments of the impairment screening system, device and method described herein utilize subject feedback from eye tests, cognitive tests, balance test and physiological activity to evaluate impairment and generate an accurate impairment in di cation. Improvements in the configuration of testing apparatus also provides a superior testing format for test administrators, while pro viding the ability to accurately record eye, cognitive, balance and physiological response for later use.

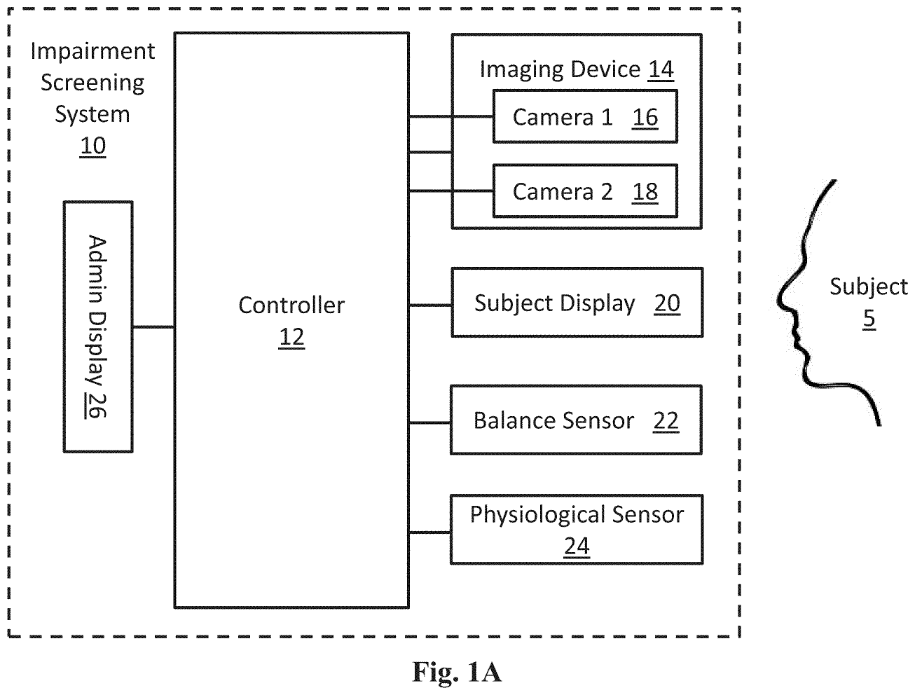

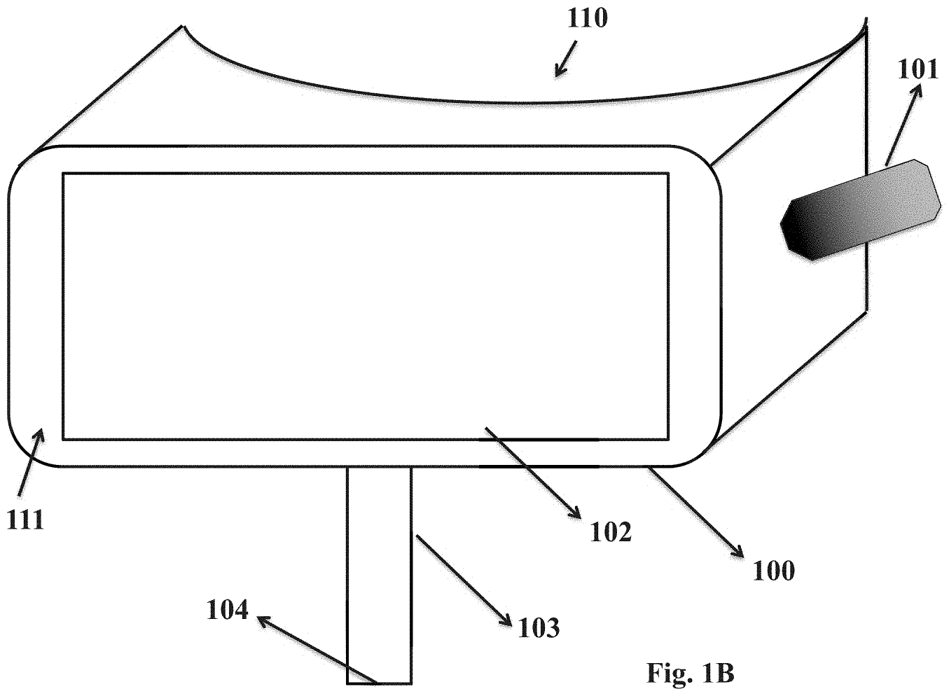

With reference now to FIG. 1A, a system 10 for screening impairment of a subject 5 according to one embodiment includes an imaging device 14 that can have a first 16 and second 18 camera for capturing images of the subject's eyes 5 during testing.

A display 20 such as an LED array or a screen is connected to a controller 12 along with the imaging device 14. The controller configured to send a first eye test signal to the imaging device 14 and a second eye test signal to the display 20 to initiate an eye test. As described in further detail below, the imaging device 14 and display 20 work in sync to conduct various kinds of tests including eye tests and cognitive tests.

The display 20 generally functions to stimulate or instruct the subject 5, while the imaging device 14 generally functions to image and evaluate the subject's response. A separate administrator display 26 can be used to provide the test administrator with feedback during testing, and can also have input functionality (such as a touch screen) to allow the administrator to setup test and access system information. Signals sent from the controller 12 to system components to initiate and conduct testing can for example be sent directly to the component, or to component sub-controllers specific to each component (or sub-groups of components) for controlling the various components. The controller 12 is configured to send a signal to initiate testing, and also receive an eye test feedback signal based on images captured of the subject's eye movement during the eye test. The controller 12 is configured to send a first cognitive test signal to the imaging device 14 and a second cognitive test signal to the display 20 to initiate a cognitive test. The controller 12 receives a cognitive test feedback signal based on captured images of subject eye movement during the cognitive test. A balance sensor 22 is connected to the controller 12, and the controller 12 is configured to receive a balance test feedback signal from the balance sensor 22 indicative of subject 5 movement during a balance test. A physiological sensor 24 is connected to the controller 12, and the controller 12 is configured to receive a physiological activity feedback signal from the physiological sensor 24 indicative of subject physiological activity. Communicative connections between components can be via hard-wired or wireless, and components can be part of modular or removable sub-systems such as a mobile device housing certain components (e.g. a smart phone or tablet housing the controller, display, sensors, a camera, etc.). The controller 12 is configured to generate an impairment indication based on the eye test feedback signal, the cognitive test feedback signal, the balance test feedback signal, and the physiological activity feedback signal. Feedback can include other types of data that can be captured by the system. For example, a camera can be used to take images of the subject's arm to detect injection sites or abnormal veins with RGB and IR images. Feedback signals can for example be cross-referenced among different tests for consistency and for comparison with known values or ranges indicative of an “impaired” or “not impaired” subject. Various devices, systems and methods for implementing this framework are described in further detail below according to the various embodiments.

FIG. 1B shows an AWI screening device according to one embodiment. The device contains a goggle-shaped frame 100 to be placed on the head and in front of the eyes which are to be positioned within a viewing cavity 110. The device includes an optional strap 101 to secure the frame on the eye, an optional screen or touch screen 102 on the front external surface 111 so the user administrating the testing can control the operation and view the results. An optional handle 11 03 is included so that the user can fix the device position in front of the head of the person being tested.

Alternatively, the handle 103 can be used to fix the device position on a station or a desk (as a base). If the strap 101 is used, the handle 103 can be ignored. The hole 104 under the handle 103 can be used as a path for wires in the case of using a hard-wired device. Alternatively, proper wireless protocols can be implemented to use the device wirelessly.

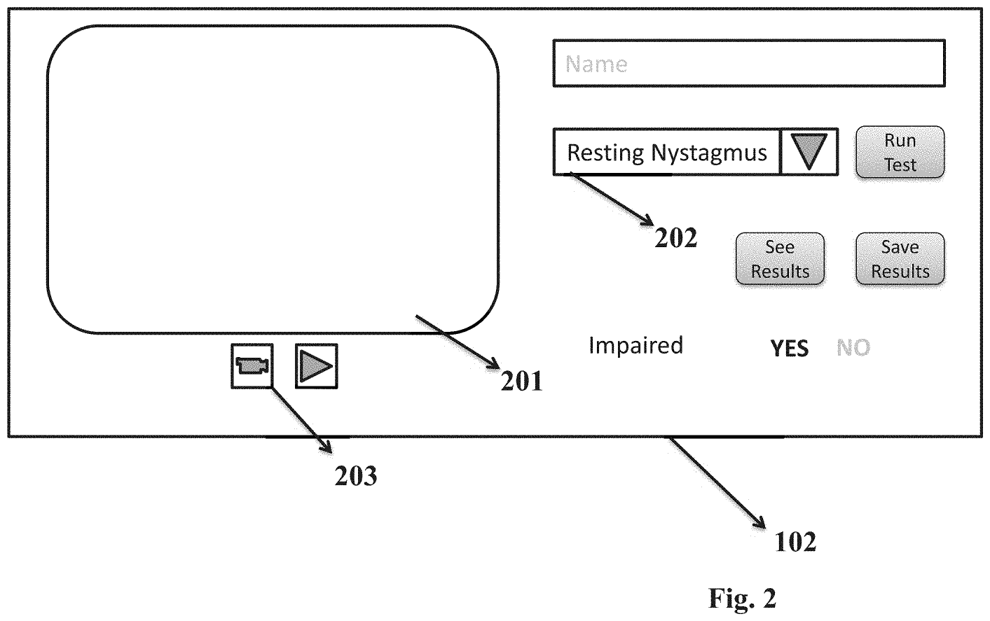

FIG. 2 schematically illustrates the screen or touchscreen of the hand-held device 102 according to one embodiment, which can optionally be used to control the operation of the impairment screening device and record and view its test results. The software on the screen 102 can allow the user to choose which tests to run. It can send commands through the communication protocol to the control system and can receive back the camera recordings in real time. The software can perform an image processing algorithm to analyze the results of the tests, and the results can be demonstrated to the user on the device screen 102. A simple schematic of software on the device is shown in FIG. 2, but the actual software user interface may vary from the shown schematic. FIG. 2 also includes the schematic of the software interface including the test selection section 202, one or two viewer sections 201 to view the real-time streamed or recorded videos, and the control buttons 203 to control the real-time stream or viewing the previously recorded videos.

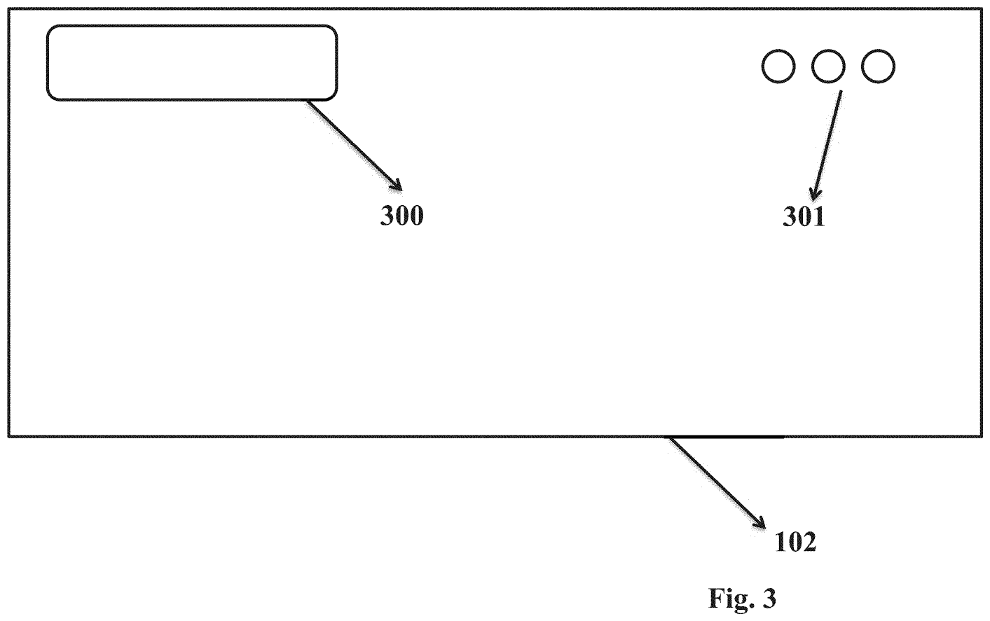

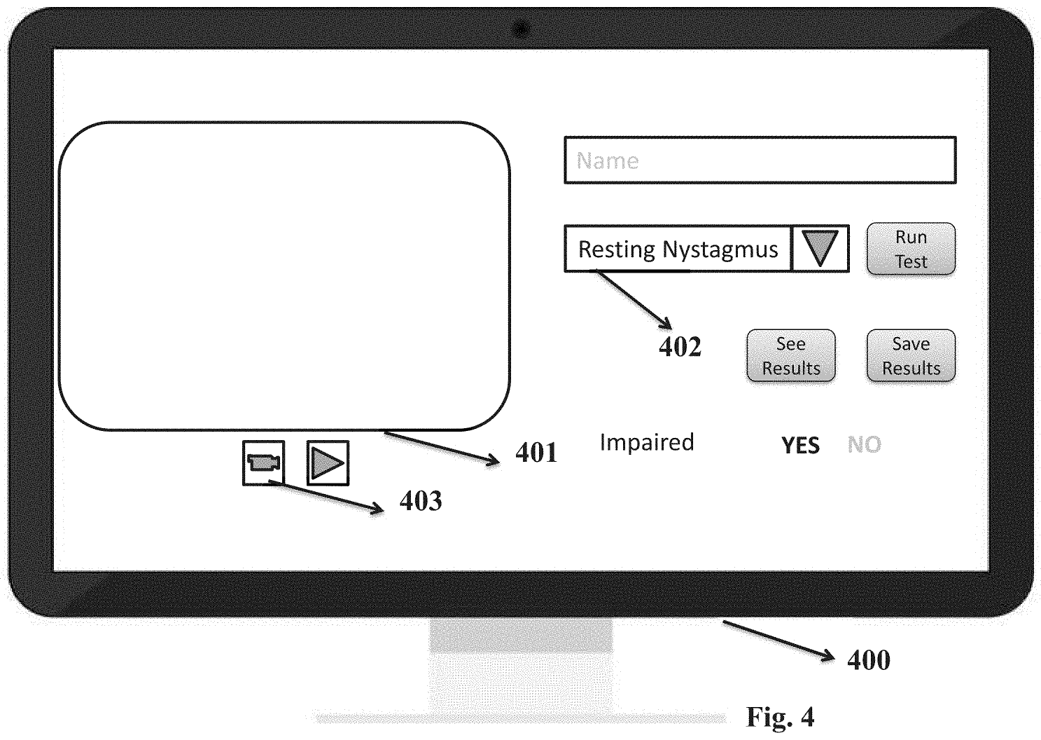

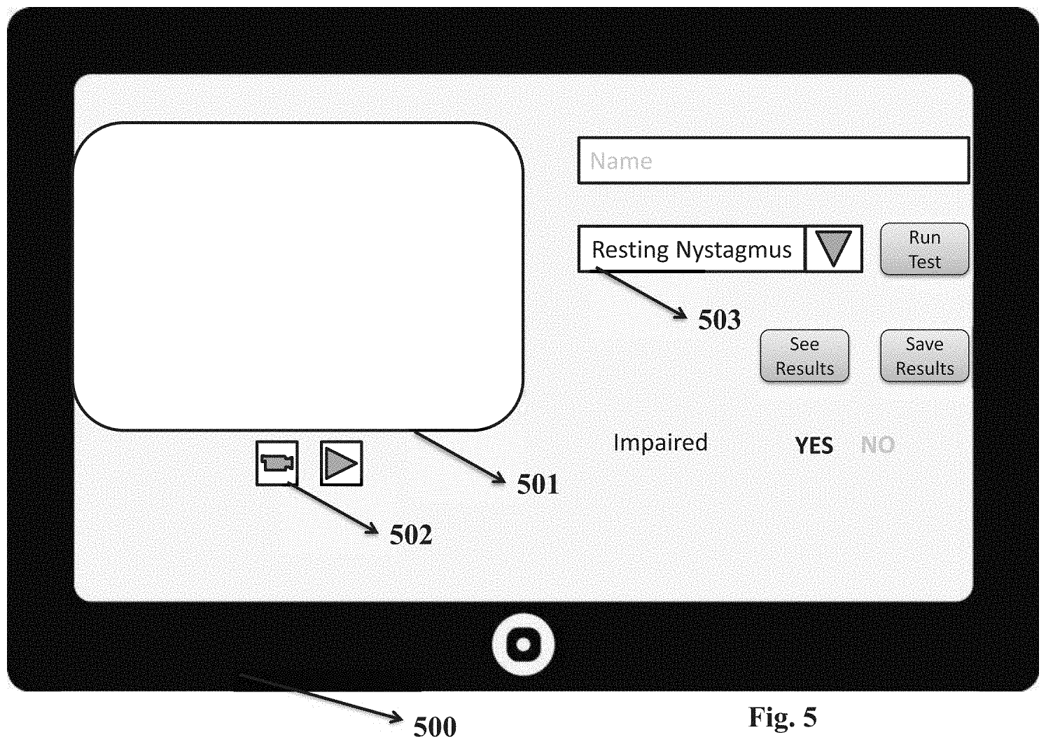

Alternatively, the impairment device might be connected to a base (wired or wireless) which is described in FIG. 4 and FIG. 5 according to various embodiments. In these cases, the device front can include a simpler screen 102 shown in FIG. 3. In this screen, the small screen 300 is used to notify the user about the status of the system by simple messaging. Alternatively, colored LED lights 301 can be used as a status indicator for the device.

The impairment screening device might be connected to a separate base station through a wired or a wireless communication. The wired communication can be, but is not limited to, USB or Ethernet. The wireless communication can be, but is not limited to, WIFI or Bluetooth communication. The base station can be a computer with the monitor 400 shown in FIG. 4 according to one embodiment, running the software interface and communicate with the user about the status of the tests and their results. Alternatively, the base station can be a hand-held device (such as a tablet) with the screen 500 shown in FIG. 5 according to one embodiment. In this case also the status of the tests, the processing, and the results demonstration will be done in the hand-held de vice. In either cases of a separate base station (computer or hand-held device), software-based cognitive tests might also be implemented to increase the accuracy of the impairment detection and its state.

FIG. 4 includes the schematic of the software interface including the test selection section 402, one or two viewer sections 401 to view the real-time streamed or recorded videos, and the control buttons 403 to control the real-time stream or viewing the previously recorded videos. FIG. 5 also includes the schematic of the software interface including the test selection section 502, one or two viewer sections 501 to view the real-time streamed or recorded videos, and the control buttons 503 to control the real-time stream or viewing the previously recorded videos.

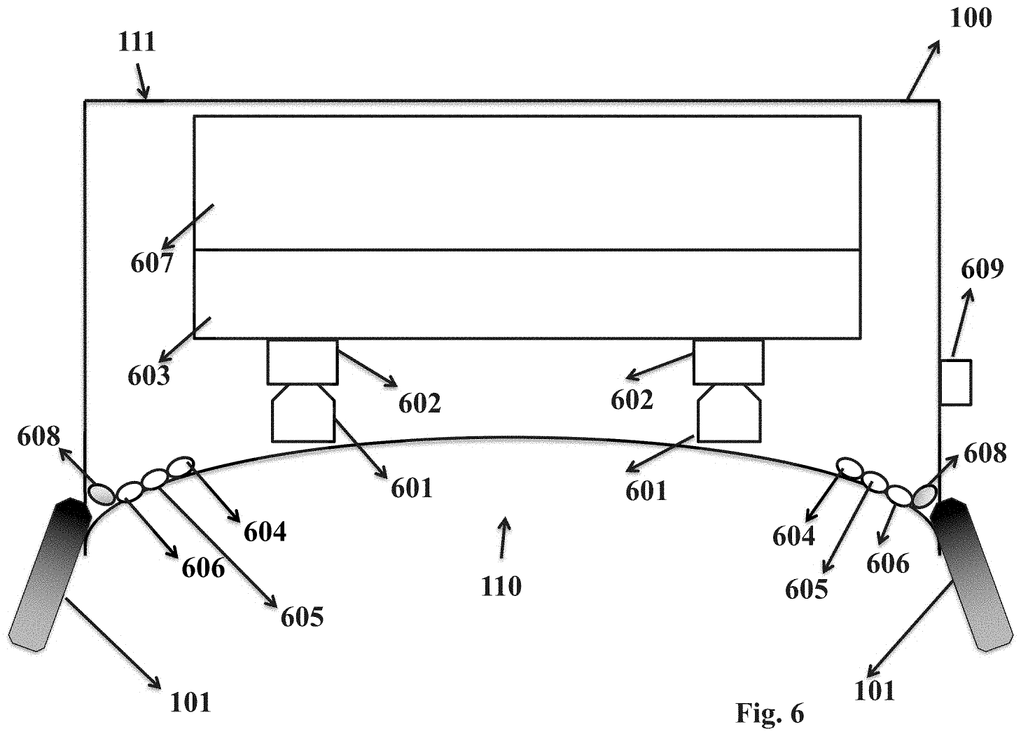

FIG. 6 demonstrates the top view of the goggle-shaped impairment screening device according to one embodiment. The batteries and the power supply board 607 are used to power up the device alternatively, the device can be connected to external power source as a replacement for the batteries and power supply board 607), and the electronics control circuit board 603 controls the operation of the device. The electronic control circuit board 603 also contains a wired or wireless communication module to transmit data between the electronic circuit board 603 and the software on the screen 102, or the computer base station 400, or the hand-held device 500.

The electronics control circuit board also can include an Accelerometer, a Gyroscope, and a Magnetometer. These three sensors can be used to perform balance test-related tests. In the head-worn or hand-held application of the impairment screening device and if the user has movement flexibility, these sensors can be used to perform related measurements and indicate the capability of the user to remain balanced and control his/her movement in different tests (including, but not limited to, one-leg stand and walk-and-run tests). The cameras 602 and all the lights are controlled via the electronic control circuit board 603 based on the test being run.

The device may also include an auditory reaction testing. In this test a beep sound is played through the speakers 608 integrated in the frame, and the user should press a button 609 after a certain amount of time (some certain seconds which will be indicated by the examiner). The user's perception of the time past is an indicator of the impairment level which is tested in this step. Alternatively, the user can use the hand controller 2400 and its buttons 2401 to react to the sound after a certain amount of time. Subject input can be collected by a variety of feedback devices besides a hand controller. For example: speech recognition can be implemented to get user responses and commands; an image processing system can be implemented to analyze different movements/reactions of the subject (e.g. raising a right hand when the subject wants to say yes and raising a left hand when the subject wants to say no); or wearable sensors such as accelerometers and gyroscopes to measure the position and movement of subject's body parts. Particularly, in the Modified Romberg (MRB} test, the head movement/position can be considered as a sign/indicator of subject's intention to continue or end the tests (e.g. counting to 30 while keeping the eyes closed and the head leaned back).

Also, the device may include extra bio signal measurement sensors as shown in FIG. 6. These sensors may include the heart-rate sensor 604, the body temperature sensor 605, and the Galvanic Skin Response (GSR) sensor, also known as Electrodermal Activity (EDA) 606. Multiple studies have shown a significant relationship between bio signal variations of human body and the state of impairment. The measurements from these sensors will be used in the software to be integrated with other test results and indicate the state of impairment with better accuracy.

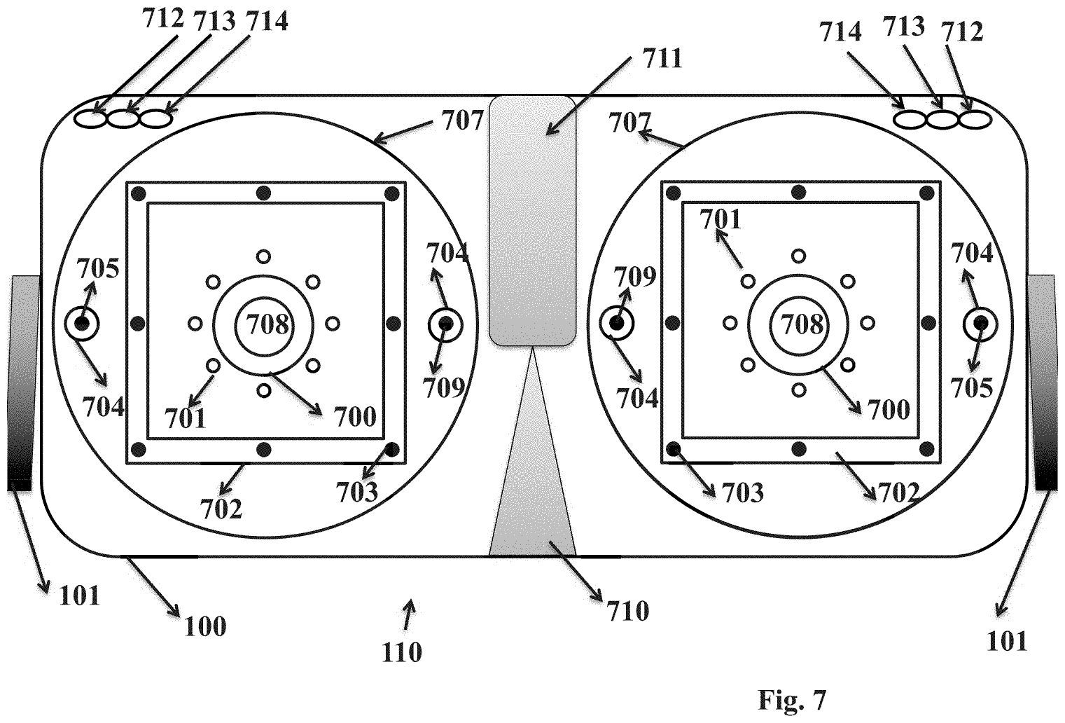

FIG. 7 demonstrates the front view of the goggle-shaped impairment screening device for the case of two separate eye spaces and cameras according to one embodiment. The front view contains two eyepieces 707 and a designated space for placing nose 710. The eyepieces are isolated from each other using a separator 711. It will be apparent to those having skill in the art that a separator 711 is not required, since embodiments disclosed herein also contemplate configurations without eyepiece isolation. In the middle of each eyepiece 707 there is a camera 708 and lens 700 system to record the eye and pupil and eyelid movements. Around the camera, one or more infrared LED(s) 701 are placed to enable the camera to record videos and take photos, as needed in the dark. The lens 700 can be a fixed focal length focused on the eye. Alternatively, an auto-focus lens 700 or a controllable focus lens 700 can be used.

Around the infrared LED(s) 701, an optional small rectangular-shaped frame 702 which covers one or more white LEDs 703 which are evenly installed on the LED-driver printed circuit board. The distribution of the white LEDs 703 can be such that each side of the rectangle will have the same and odd number of white LEDs. The optional rectangular-shaped frame 702 has small! holes in the place of white LEDs 703, so the light of the LEDs will be pin-shaped.

Also, in the design of FIG. 7, separate white LEDs 705, 709 may be used to perform a more comprehensive testing. These LEDs 705, 709 may be also optionally cove red with a cover 704 so their emitted light is pin-shaped. In one embodiment, LEDs are RGB LEDs so that color blindness can be identified if needed. In one embodiment, a laser system/projector to project light (e.g. a moving dot) is implemented on the inner surface of the goggle for an eye tracking test. In one embodiment, the position of the LEDs has a field of view of 90 degrees in either side. In one embodiment, the display construction is configured to create curved LED arrays. Two or more LED arrays next to each other can also form different angles.

Also, in the design of the device front, the bio signal sensors including, but not limited to, heart rate sensor 714, temperature sensor 713, and the GSR sensor 712, are placed such that they will touch the user skin. The heart rate sensor 714 may be working based on an infrared transmitter and receiver technology to pick up the sudden changes in the blood flow. The temperature sensor 713 may be working based on infrared temperature sensing technology or resistive temperature sensing electrodes.

The GSR sensor 712 may be working based on solid conductive electrodes and determining the variations of the conductance of a small electrical signal through the skin.

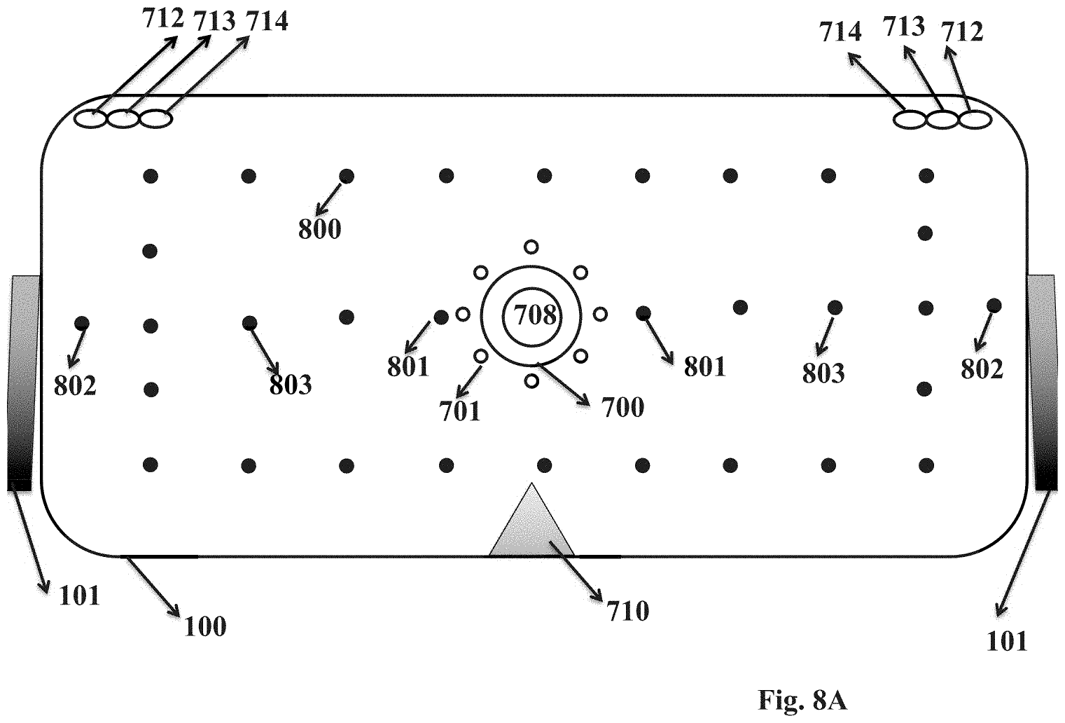

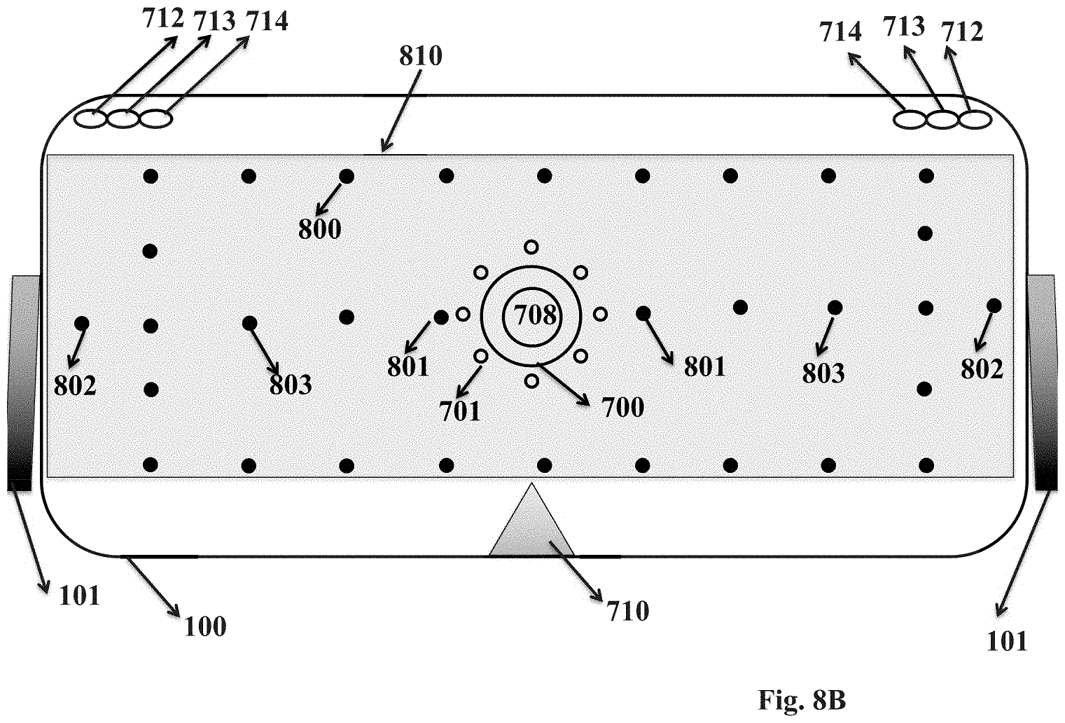

Alternatively, FIG. 8 demonstrates the front view of the goggle-shaped impairment screening device for the case of one eye space and one or more cameras. The front view contains one eye space and a designed space for placing nose 710. In the middle of the eye space, there is a camera 708 and lens 700 system to record the eye and pupil and eyelid movements. Around the camera, one or more Infrared LED(s) 701 are placed to enable the camera to record videos and take photos, as needed, in the dark. The lens 700 can be a fixed focal length focused on the eye. Alternatively, an auto-focus lens 700 or a controllable focus lens 700 can be used.

In the design of FIG. 8A, eye tests may be running with flexibility such that a smoother light variation can be simulated by turning the white LED rows 800 on and off. The number of white LEDs in the rows 800 or the number of horizontal or vertical rows can be one or more. Moreover, this design may also have extra white LEDs in different locations, specifically, closer to nose LEDs 801, center LEDs 803, and maximum deviation LEDs 802. Each LED location may be used for a separate testing condition. In one embodiment as shown in FIG. 88, and optical diffuser 810 is configured to cover the LEDs so that light motion appears to the subject to move along a smoother path. This way, the subject does not stop to focus on individual lights as they illuminate, and the system is less prone to detecting a false-positive jerking eye movement of a non-impaired subject stopping to focus on individual lights. Electrically or manually adjustable lenses can be implemented to help change the perception of distance when a subject. is being tested by the goggle. This is beneficial for the subset of subjects that cannot clearly see or follow objects that are very close to their eyes. By using those lenses and virtually changing the distance of the light stimuli from the subject's eyes, the tests can be performed accurately, and the light stimuli can be clearly viewed.

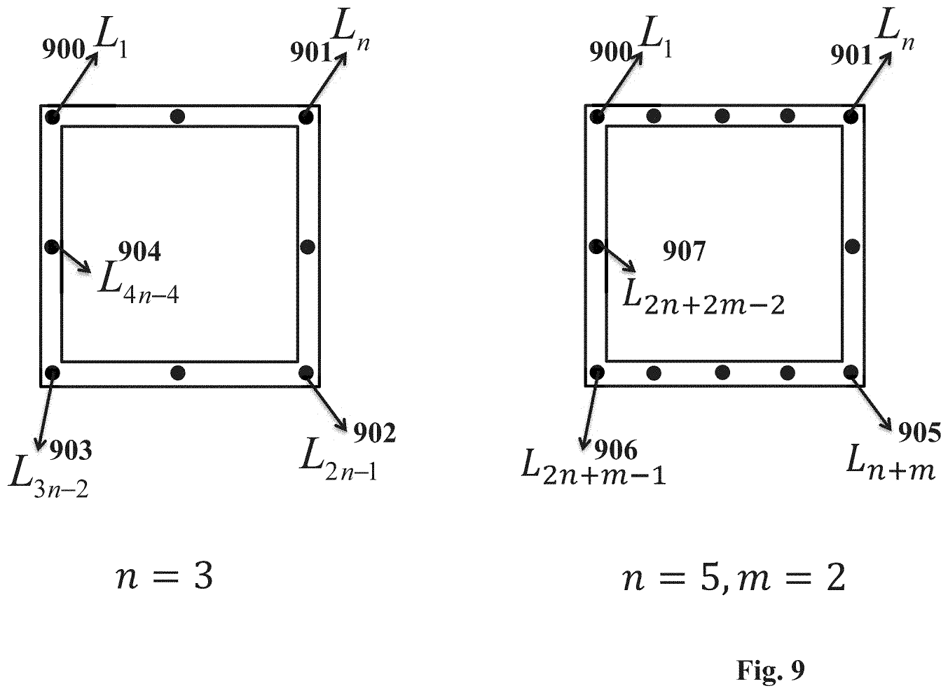

The white LEDs in each design can vary in numbers. Color LEDs can also be utilized. FIG. 9 demonstrates two possible distributions (one for each design) but in general the design is not limited to the number of LEDs shown. Also, FIG. 9 shows the naming convention for each white LED depending on the design; the left image is for each one of the two eye pieces in the two eye space design (FIG. 7) and the right image is for the design in the case of the one eye space system (FIG. 8). In the case of two eye space design the number of white LEDs in each side may be equal, while in the case of one eye space they are different. L₁ 900 shows the white LED on the top left corner. Lₙ 901 shows the white LED on the top right corner. Based on this there should be always n white LEDs on each side of the LED-driver printed circuit board, which the value of n can be any odd number larger than 3. Also, in this naming convention, the white LED in the bottom right corner is named L₂ₙ₋₁ 902 in the case of two eye space design and is named Lₙ₊ₘ 905 in the case of one eye space design, the LED in the bottom left corner is named LJn-2 903 in the case of two eye space design and is named L₂ₙ₊ₘ₋₁ 906 in the case of one eye space design, and the last LED in the loop (one below the top left corner) is named L₄ₙ₋₄ 904 in the case of two eye space design and is named L₂ₙ₊₂ₘ₋₂907 in the case of one eye space design. Therefore, the total number of white LEDs will be 4n−4 in the case of two eye space design and is 2n+2m−2 in the case of one eye space design.

Two or more white LEDs 705, 709 are located at each side of each eye, covered by a frame with a small hole 704 to make a pin-shaped light. These LEDs 705, 709 are also being controlled individually depending on the test being performed.

Alternatively, same LEDs are included in FIG. 8 as 801, 802.

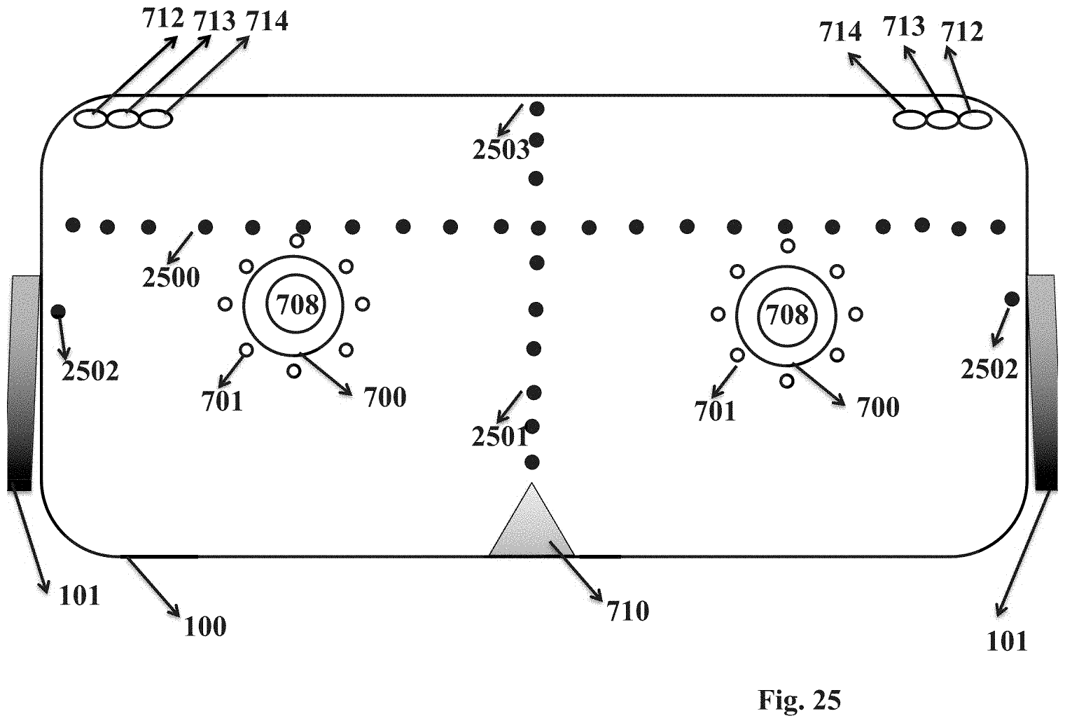

Alternatively, the embodiment in FIG. 25 demonstrates another design to perform the eye tests. The device consists of one or two cameras 708, one or two lens systems 700, one or more infrared LEDs 701 per camera, a horizontal row of white LEDs 2500, a vertical row of white LEDs 2501, two white LEDs at maximum deviation 2502 and one white LED at maximum top position 2503. All the tests reported below can be also translated into this design.

The presented impairment screening device along with its software, can perform one or more of 19 different tests related to impairment (discussed below). The user will have access to run individual tests and view their individual or integrated results. The results of individual tests may be integrated with specific integration algorithms to result in better accuracy of impairment screening.

Cognitive tests: The cognitive tests may be running on the user interface of the device if the device is using a base station to control the activities. In these cognitive tests, the user will be tasked to follow certain instructions to diagnose the capability of the user to follow and to track certain movements on the screen. Also, some of the cognitive tests are mentioned below.

Physiological measurement tests: The bio signal measurement tests may be done using the bio signal sensors including, but not limited to, heart rate sensor 714, temperature sensor 713, and the GSR sensor 712. These signals may be recorded continuously or discontinuously in real-time and the raw data will be transferred to the control circuit and/or the base station for filtering and analysis.

Balance tests: Balance tests including, but not limited to, the One-Leg-Stand test and Walk-and-Run test (as the two most common types in field sobriety tests) can be performed by tasking the user to follow a routine (as required by the tests) while wearing the impairment screening device. The movement of the user's head will be recorded using the embedded accelerometer, gyroscope, and the magnetometer, giving 9 degrees-of-freedom for measuring the user activity and balance. The results of measurements from one or more of the sensors may be integrated to determine the balance of the user. Also, in the area of Balance tests, other tests such as force-detecting insoles or shoes, gait and posture detection technologies, and wearable devices may be considered to increase the accuracy of balance detection. Specifically, a few balance tests are described below. To detect balance and body position, smart cameras (or depth cameras) can be used with or in place of sensors (e.g. Intel RealSense cameras or other similar technologies) to record RGB videos and depth information while the subject is performing various tests (e.g. walk-and-turn or one-leg-stand tests). The recorded videos are analyzed to find the position of each part of the subject's body in a 30 space. Accordingly, movements, balance, and the level of shakiness can be quantified and compared with preset values or previously recorded values.

Eye tests: In all the eye tests performed by the impairment screening device, the cameras 708, and the infrared LEDs 701 may always have turned on before the start of the test. Also, the camera controlling software is set such that it automatically records videos of eye movement and will stream the video in real time to the software installed on the device itself, or on the computer base station 400, or the hand-held device 500. The image processing algorithms may run automatically on the recorded videos and will demonstrate the results related to the test being performed.

The final eye test results may be integrated with other tests performed to increase the accuracy of the impairment screening.

The integration algorithms are outside of the scope of this patent. Details of certain eye tests, cognitive tests, balance tests and physiological feedbacks that can be relied upon to make an impairment determination are as follows:

Eye Test 1: Resting Nystagmus

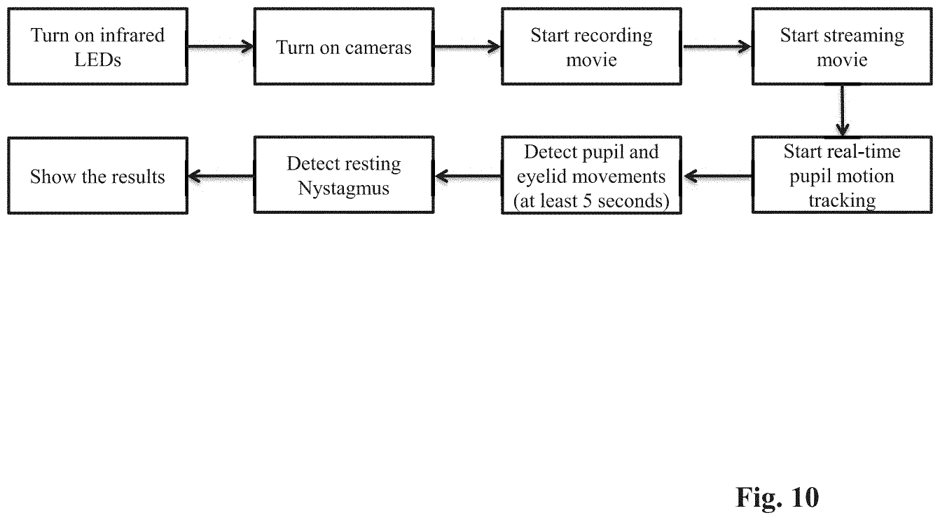

This test is being done by keeping the eyepieces 707 in dark (alternatively being done with the one eye space setup) and recording videos of the movement of the eye. The steps of this test are shown in FIG. 10. Test 1 is designed based on the fact that in some cases of the person being under influence of drugs or alcohol, at resting position of eye, the pupils will have sudden and jerky movements. Utilizing the image processing algorithms for this test, the position of the pupil may be tracked, so that any movements may be recorded and used to determine that if the person has the resting Nystagmus condition or not.

Eye Test 2: Eyelid Twitching

The steps of this test are also shown in FIG. 10 as this test does not require a separate testing condition and can be integrated with any of the other eye tests. This test is based on the observation of eyelid twitching in a significant number of impairment cases. Therefore, the movement of the eyelid may be recorded using the device and maybe analyzed utilizing image processing algorithms for automated detection of twitching.

Eye Test 3: Horizontal Gaze Nystagmus (HGN)

In the case of the two eye spaces design (FIG. 7), this test is being done by keeping the eyepieces 707 in dark initially and recording videos of the movement of the eye. Then, the white LED L1 900 will be turned on for 1/n seconds. Then the second white LED L2 will be turned on for 1/n seconds. This will repeat until at the end of the 1-second period of time, white LED Ln 901 will be turned on. Overall during the last 1 second white LEDs on the top row will be turned on and off one-by-one starting from the left.

During the next step the bottom white LEDs will be turned on and off one-by-one starting from the right with the time periods of 1/n seconds. During both steps the camera 708 will record videos or photos of the pupil's movement while the infrared LEDs 701 are on at all times.

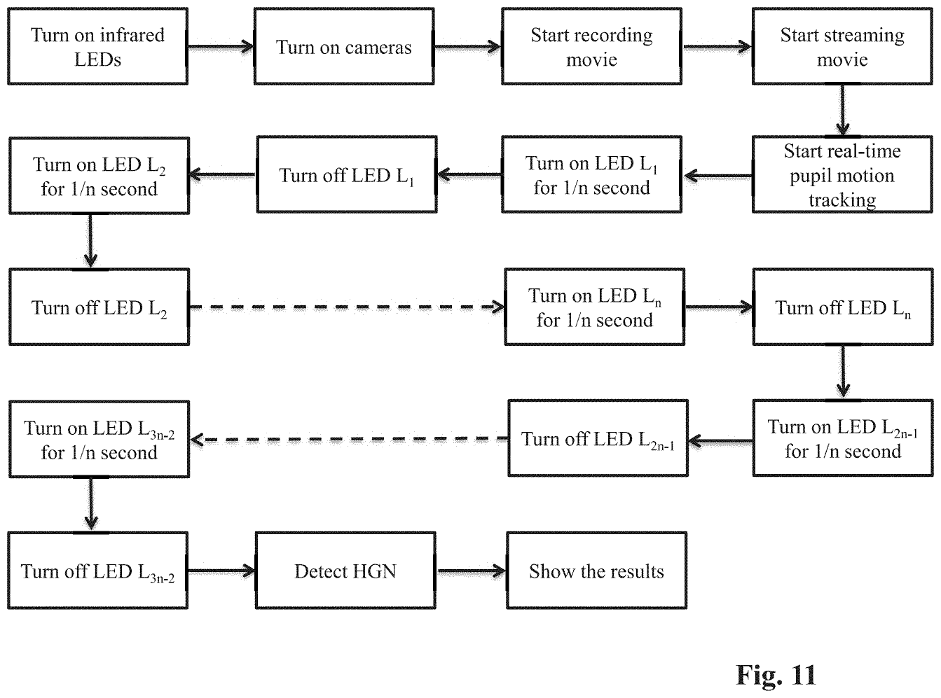

The steps of this test are shown in FIG. 11. This test is designed based on the fact that in some cases of the person being under influence of drugs or alcohol, the pupil cannot follow the objects horizontally and will have sudden or jerky movements. Utilizing the image processing algorithms for this test, the position of the pupil is tracked, so that any movements will be recorded and will be used to determine that if the person has the HGN condition or not.

The steps shown in FIG. 11 may be performed for both eyes simultaneously or one-by-one individually.

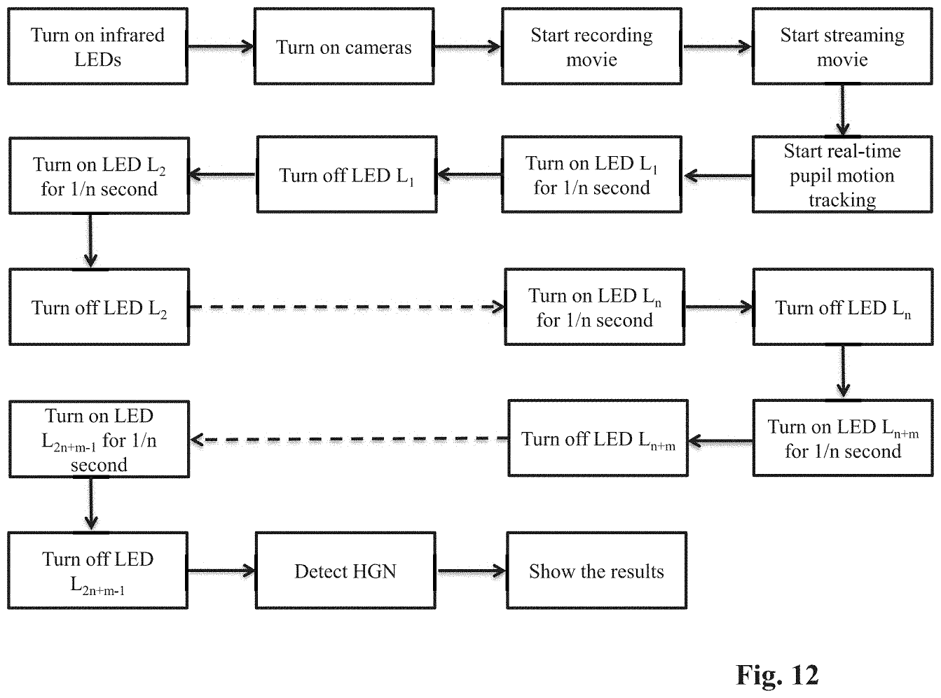

In the case of the one eye space design (FIG. 8), this test is being done by keeping the eye space in dark initially and recording videos of the movement of the eye. Then, the white LED L1 900 will be turned on for l/n seconds. Then the second white LED L2 will be turned on for 1/n seconds. This will repeat until at the end of the 1-second period of time, white LED Ln 901 will be turned on. Overall during the last 1 second white LEDs on the top row will be turned on and off one-by-one starting from the left. During the next step the bottom white LEDs will be turned on and off one-by-one starting from the right with the time periods of 1/n seconds. During both steps the camera 708 will record videos or photos of the pupil's movement while the infrared LEDs 701 are on at all times. The steps of this test are shown in FIG. 12.

In the embodiment of FIG. 25, the HGN tests will be starting at the middle white LED in the horizontal row 2500. The turning on/off sequence will then go to the left or right in a smooth manner (predefined steps of time), until it reaches the white LED at maximum deviation 2502. It will then rest at that white LED for a predefined and fixed amount of time and then the sequence will be reversed, and the sequence will go to the right or left side in a smooth manner with a predefined step of time until it reaches the other white LED at maximum deviation 2502. It will then rest at that white LED for a predefined and fixed amount of time and then the sequence will be reversed, and the sequence will go to the left or right side again in a smooth manner to reach to the middle white LED in the horizontal row 2500. This loop can be iterated for one or more times. The videos of the eye movement will be captured and sent to the base station for further analysis.

Eye Test 4: Vertical Gaze Nystagmus (VGN)

This test is being done by keeping the eyepieces 707 in dark initially and recording videos of the movement of the eye. Then, the white LED L₁ 900 will be turned on for 1/n seconds. Then the second white LED L₄ₙ₋₄ 904 will be turned on for 1/n seconds. This will repeat until at the end of the 1-second period of time, white LED Lan-2 903 will be turned on. Overall during the last 1 second white LEDs on the left will be turned on and off one-by-one starting from the top.

During the next step the right white LEDs will be turned on and off one-by-one starting from the bottom with the time periods of 1/n seconds. During both steps the camera 708 will record videos of the pupil's movement while the infrared LEDs 701 are on at all times.

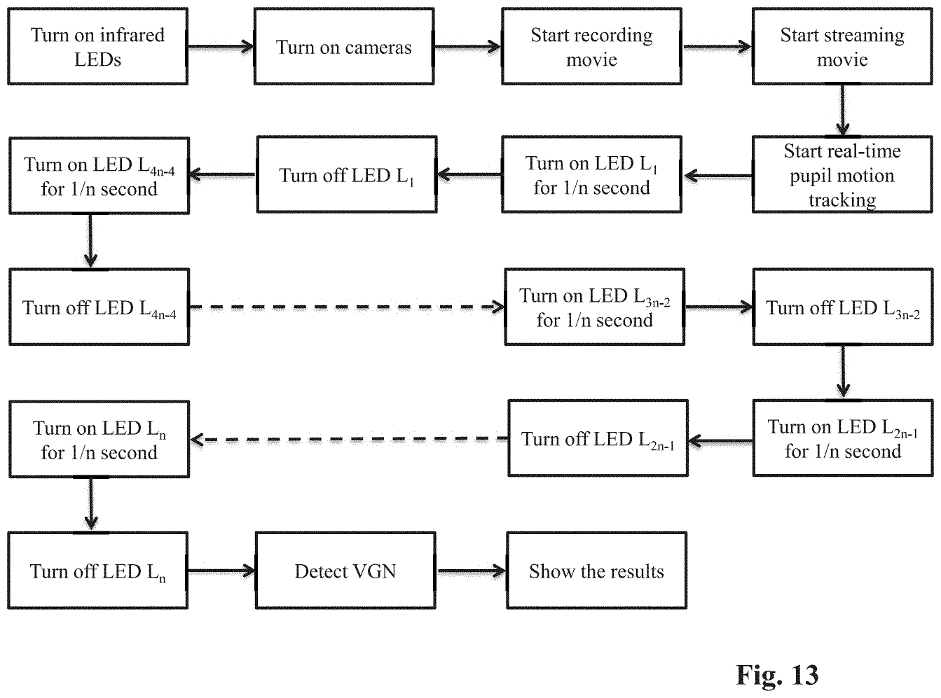

The steps of this test are shown in FIG. 13. This test is designed based on the fact that in some cases of the person being under influence of drugs or alcohol, the pupil cannot follow the objects vertically and will have sudden or jerky movements. Utilizing the image processing algorithms for this test, the position of the pupil is tracked, so that any movements will be recorded and will be used to determine that if the person has the VGN condition or not.

The steps shown in FIG. 13 may be performed for both eyes simultaneously or one-by-one individually.

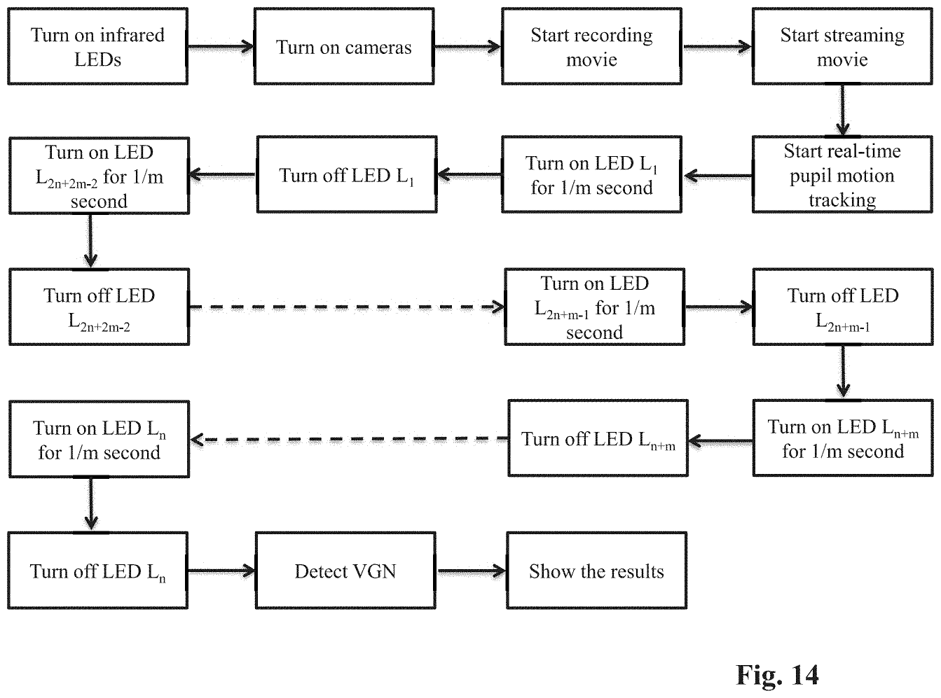

In the case of the one eye space design (FIG. 8), this test is being done by keeping the eye space in dark initially and recording videos of the movement of the eye. Then, the white LED L1 900 will be turned on for 1/n seconds. Then the second white LED L₂ₙ₊₂ₘ₋₂ 907 will be turned on for 1/n seconds. This will repeat until at the end of the 1-second period of time, white LED L₂ₙ₊ₘ₋₁ 906 will be turned on. Overall during the last 1 second white LEDs on the left will be turned on and off one-by-one starting from the top. During the next step the right white LEDs will be turned on and off one-by-one starting from the bottom with the time periods of 1/n seconds. During both steps the camera 708 will record videos of the pupils movement while the infrared LEDs 701 are on at all times. The steps of this test are shown in FIG. 14.

In the embodiment of FIG. 25, the VGN tests will be starting at the middle white LED in the vertical row 2501. The turning on/off sequence will then go up in a smooth manner (predefined steps of time), until it reaches the white LED at top 2503. It will then rest at that white LED for a predefined and fixed amount of time and then the sequence will be reversed, and the sequence will go to the bottom side in a smooth manner with a predefined step of time until it reaches the other white LED at the bottom. It will then reverse the sequence will go up again in a smooth manner to reach to the middle white LED in the vertical row 2501. This loop can be iterated for one or more times. The videos of the eye movement will be captured and sent to the base station for further analysis.

Eye Test 5: Equal Pupils

This test is being done by keeping the eyepieces 707 in dark initially (alternatively being done with the one eye space setup) and recording videos of the movement of the eye. The steps of this test are shown in FIG. 15. Test 5 is designed based on the fact that in some cases of the person being under influence of drugs or alcohol, the pupil sizes of eyes are different. Utilizing the image processing algorithms for this test, the size of the pupils are measured, so that any difference will be recorded and will be used to determine that if the person has the not-equal pupils condition or not.

Eye Test 6: Lack of Smooth Pursuit

This test is being done by keeping the eyepieces 707 in dark initially and recording videos of the movement of the eye. Then, the white LED L₁ 900 will be turned on for 1/n seconds. Then the second white LED L₂ will be turned on for 1/n seconds.

This will repeat until at the end of the (4n−4)/n-second period of time, white LED L₄ₙ₋₄ 904 will be turned on. Overall during the last (4n−4)/n seconds, all white LEDs will be turned on and off one-by-one starting from the top left.

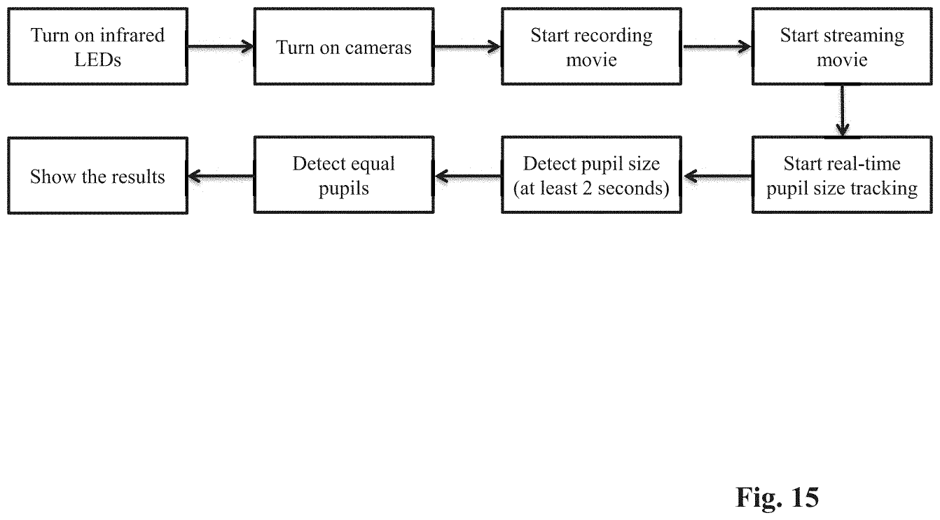

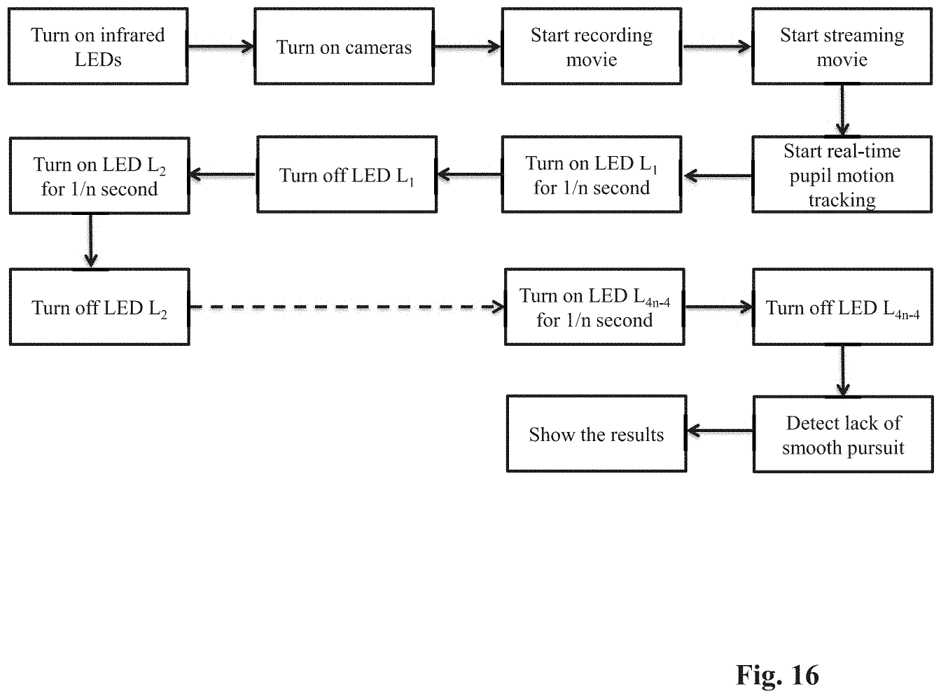

The steps of this test are shown in FIG. 16. This test is designed based on the fact that in some cases of the person being under influence of drugs or alcohol, the pupil cannot smoothly pursuit the moving objects and will have non-smooth movements. Utilizing the image processing algorithms for this test, the position of the pupil is tracked, so that any movements will be recorded and will be used to determine that if the person has the Lack of Smooth Pursuit condition or not.

The steps shown in FIG. 16 may be performed for both eyes simultaneously or one-by-one individually.

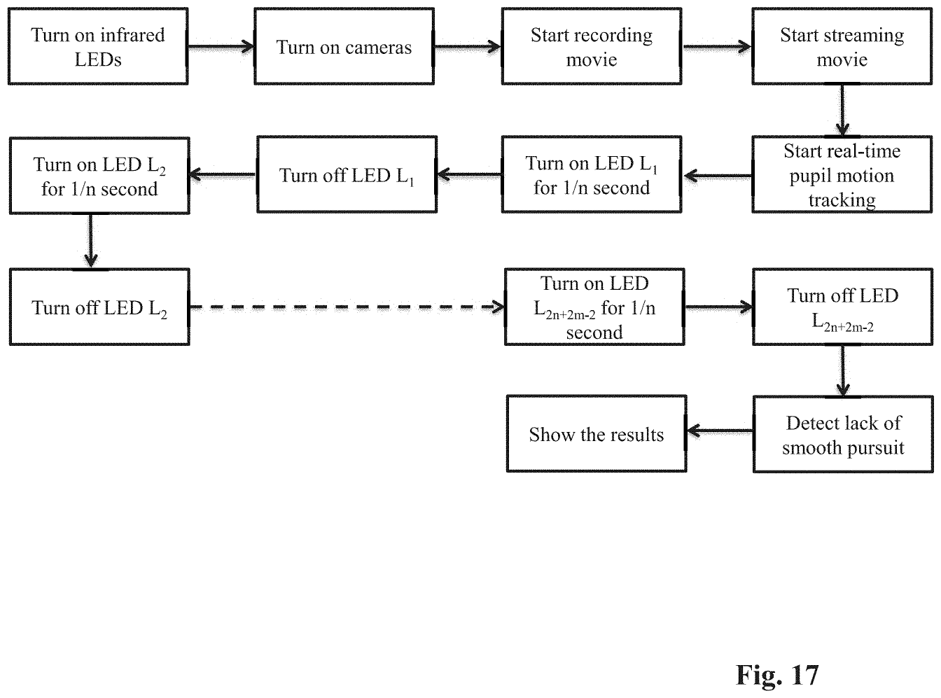

In the case of the one eye space design (FIG. 8), this test is being done by keeping the eye space in dark initially and recording videos of the movement of the eye. Then, the white LED L1 900 will be turned on for 1/n seconds. Then the second white LED L₂ will be turned on for 1/n seconds. This will repeat until at the end of the period of time, white LED L₂ₙ₊₂ₘ₋₂ 907 will be turned on. The steps of this test are shown in FIG. 17.

Eye Test 7: Nystagmus at Maximum Deviation

This test is being done by keeping the eyepieces 707 in dark initially and recording videos of the movement of the eye. Then, the right white LED 705 will be turned on for 2 seconds and will be turned off after that. After 1 second of resting, the left white LED 705 will be turned on for 2 seconds and will be turned off after that.

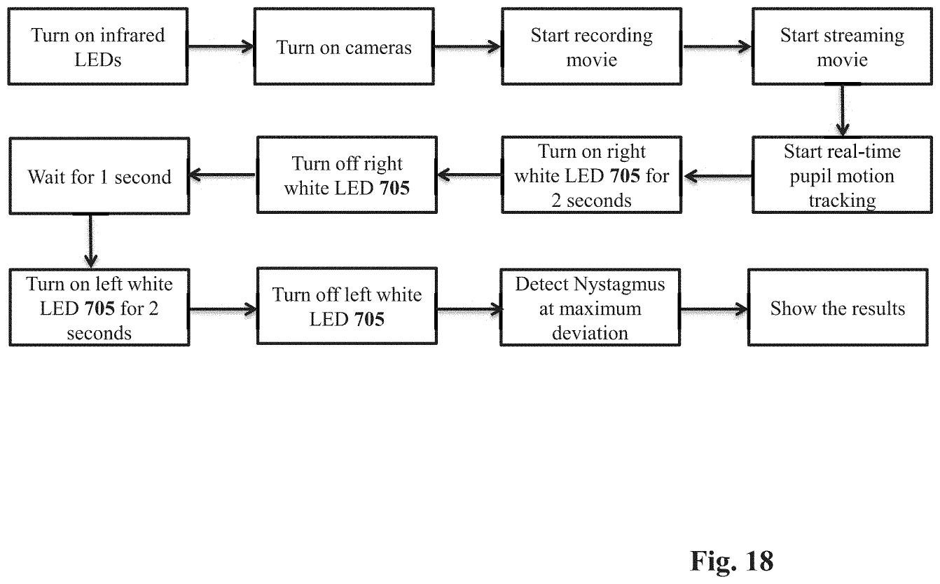

The steps of this test are shown in FIG. 18. Test 7 is designed based on the fact that in some cases of the person being under influence of drugs or alcohol, at maximum deviation of eyes, the pupils will have sudden and jerky movements. Utilizing the image processing algorithms for this test, the position of the pupil is tracked, so that any movements will be recorded and will be used to determine that if the person has the Nystagmus at Maximum Deviation condition or not.

The steps shown in FIG. 18 will be performed for each eye individually.

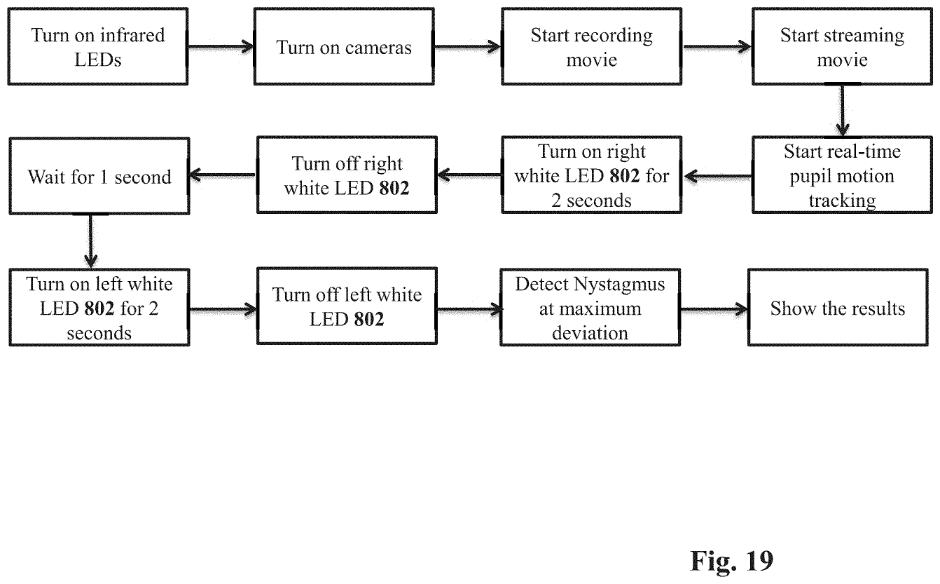

In the case of the one eye space design (FIG. 8), this test is being done by keeping the eye space in dark initially and recording videos of the movement of the eye. Then, the right white LED 802 will be turned on for 2 seconds and will be turned off after that. After 1 second of resting, the left white LED 802 will be turned on for 2 seconds and will be turned off after that. The steps of this test are shown in FIG. 19.

Eye Test 8: Nystagmus Prior to 45 Degrees

This test is being done by keeping the eyepieces 707 in dark initially and recording videos of the movement of the eye. Then, the white LED in the middle of the left side will be turned on for 2 seconds and will be turned off after that. After 1 second of resting, the white LED in the middle of the right side will be turned on for 2 seconds and will be turned off after that.

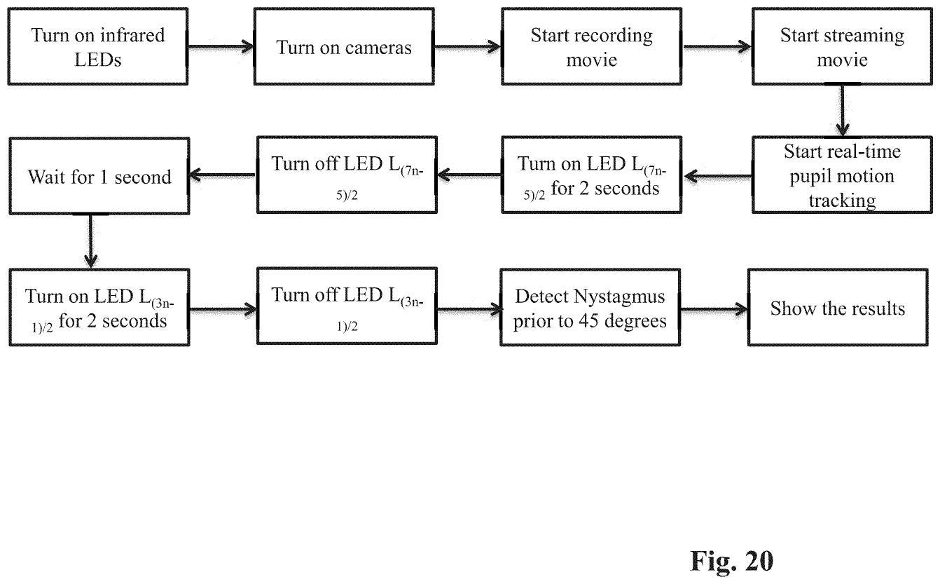

The steps of this test are shown in FIG. 20. Test 8 is designed based on the fact that in some cases of the person being under influence of drugs or alcohol, at 45-degrees deviation of eyes, the pupils will have sudden and jerky movements. Utilizing the image processing algorithms for this test, the position of the pupil is tracked, so that any movements will be recorded and will be used to determine that if the person has the Nystagmus Prior to 45 Degrees condition or not.

The steps shown in FIG. 20 will be performed for each eye individually.

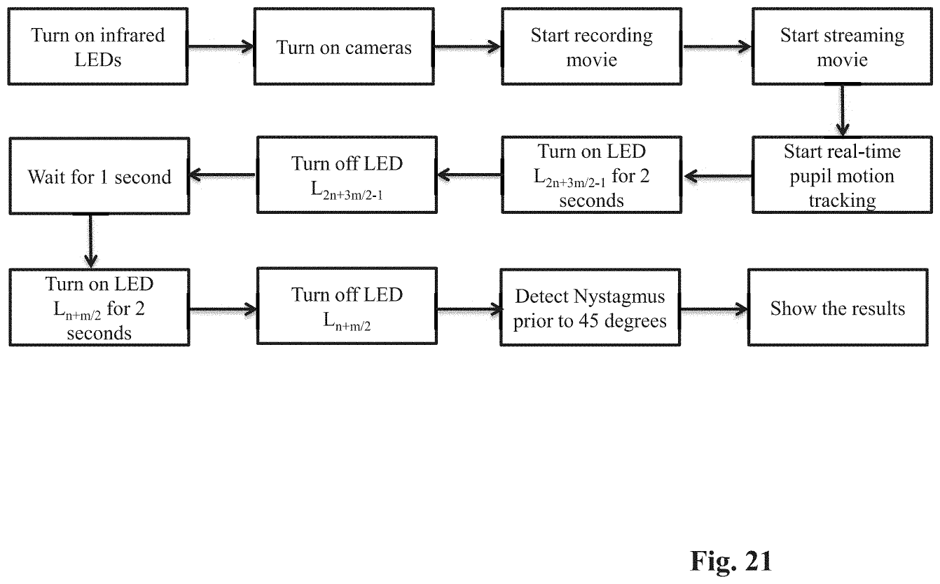

In the case of the one eye space design (FIG. 8), this test is being done by keeping the eye space in dark initially and recording videos of the movement of the eye. Then, the white LED in the middle of the left side will be turned on for 2 seconds and will be turned off after that. After 1 second of resting, the white LED in the middle of the right side will be turned on for 2 seconds and will be turned off after that. The steps of this test are shown in FIG. 21.

Eye Test 9: Non-Convergence

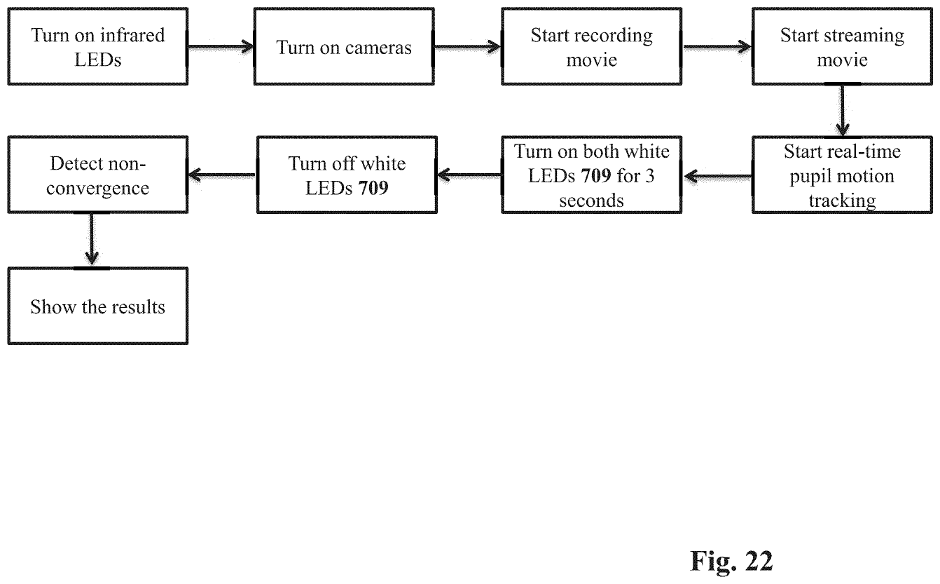

This test is being done by keeping the eyepieces 707 in dark initially and recording videos of the movement of the eye. Then, the white LEDs 709 will be turned on for 3 seconds (for both eyes simultaneously) and then turned off. The steps of this test are shown in FIG. 22.

Test 9 is designed based on the fact that in some cases of the person being under influence of drugs or alcohol, the eyes will have the issue of not being able to converge to the same point. Utilizing the image processing algorithms for this test, the position of the pupil is tracked, so that any movements will be recorded and will be used to determine that if the person has the Non-convergence condition or not.

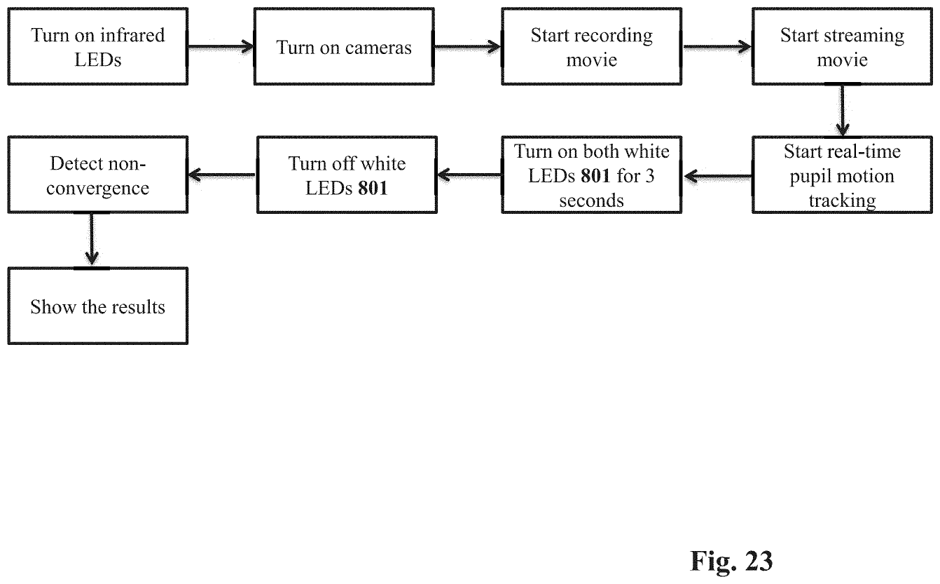

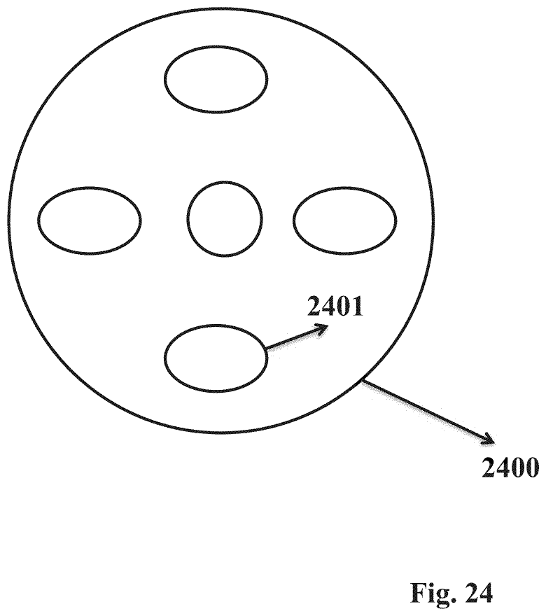

In the case of the one eye space design (FIG. 8), this test is being done by keeping the eye space in dark initially and recording videos of the movement of the eye. Then, the white LEDs 801 will be turned on for 3 seconds (for both eyes simultaneously) and then turned off. The steps of this test are shown in FIG. 23.

Balance Test 10: Walk and Turn Test

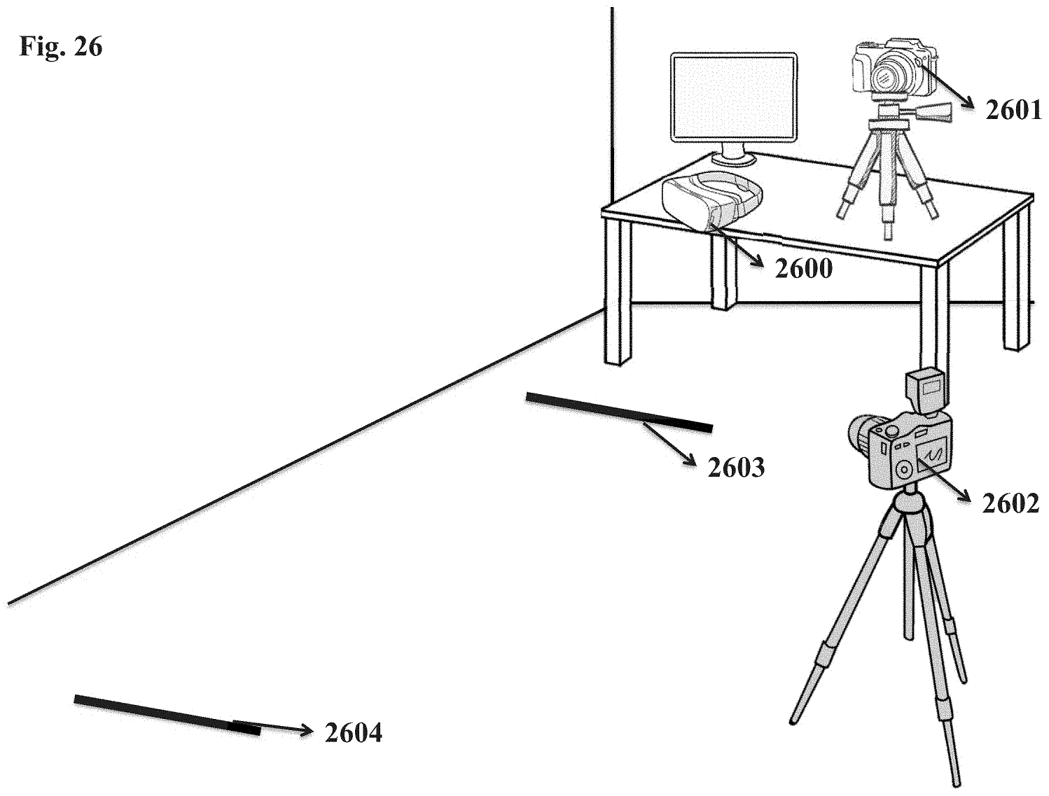

Test 10 will be performed similar to the standard field sobriety test. The subject will be asked to start from the Start Line 2604 (see FIG. 26) and walk slowly and in a heel-to-toe manner (9 steps) to the Finish Line 2603 back and forth. The subject's movement will be recorded using one or more cameras 2601 and 2602. The cameras can be simple digital cameras. Alternatively, the cameras 2601 and 2602 can also include the depth camera technology to detect depth of each pixel. The information from the cameras 2601 and 2602 can be used to detect the clues under the standard field sobriety test as well as other balance-related measures. Balance sensors such as an accelerometer, gyroscope, magnetometer, shoe or insole force sensor, or wearable activity monitoring sensor can also be utilized.

Balance Test 11: One Leg Stand Test

Test 11 can be performed similar to the standard field sobriety test. The subject will be asked to stand on one leg when the other leg is up, in front, with a certain distance from the ground. The subject's movement will be recorded using one or more cameras 2601 and 2602. The cameras can be simple digital cameras. Alternatively, the cameras 2601 and 2602 can also include the depth camera technology to detect depth of each pixel. The information from the cameras 2601 and 2602 can be used to detect the clues under the standard field sobriety test as well as other balance-related measures.

Balance Test 12: Finger to Nose Test

Test 12 will be performed similar to the standard field sobriety test. The subject will be asked to touch the nose while maintaining the 90 degrees angle for the arms and elbow for one or more times. The subject's movement will be recorded using one or more cameras 2601 and 2602. The cameras can be simple digital cameras. Alternatively, the cameras 2601 and 2602 can also include the depth camera technology to detect depth of each pixel. The information from the cameras 2601 and 2602 can be used to detect the clues under the standard field sobriety test as well as other balance-related measures.

Cognitive Test 13: Time Perception Test

The time perception test is being performed with playing a beep in one or both speakers 608 (FIG. 6) or with any other visual (turning on the white LEDs) or auditory signals (commanding the subject verbally), and the user is being asked to push the button 609 or one of the hand controller buttons 2401 after a fixed amount of time (counting seconds). The user's perception of the time past has a direct relationship with the level of impairment. The exact time difference between the beep sound and pressing of the button is measured and will be stored.

Physiological Activity Test 14: Elevated Heart Rate Test:

This test is being performed simply by using the heart rate sensor 604 or any other similar measurement systems. If the heart rate is more than the normal (standard average values) then the test procedure may raise a flag.

Balance Test 15: Modified Romberg Test

Test 15 will be performed similar to the standard field sobriety test. The subject will be asked to tilt the head backwards (while wearing the goggles 2600), close the eyes, and count a defined period of time (for example 30 seconds). The subject's movement will be recorded using one or more cameras 2601 and 2602. The cameras can be simple digital cameras. Alternatively, the cameras 2601 and 2602 can also include the depth camera technology to detect depth of each pixel. The information from the cameras 2601 and 2602 can be used to detect the clues under the standard field sobriety test as well as other balance-related measures. Also, the eyelid movement will be recorded by the goggles 2600 cameras and can be transferred to the base station for further analysis.

Balance Test 16: Head Movement and Jerk Test

Test 16 will be performed as an embedded test in all pother tests, as the movement of the head is recorded using Accelerometer and Gyroscope integrated in the electronic circuit board and can be analyzed later in order to find unusual and/or jerky movements.

Cognitive Test 17: Reaction Time Test

Test 17 will be performed by asking the subject to push a button 2401 in the hand controller 2400 (wired or wireless) as soon as they see a white LED turning on. This test will measure the subject's reaction time. The reaction time (time period between turning the white LED on and the user pushing the button) will be recorded in milliseconds and can be used as a measure of impairment. The test will be done multiple time and the LEDs will be selected randomly.

Cognitive Test 18: Digit Vigilance Test

Test 18 will be performed using the screen at the base station. During this test, subject is required to press the YES button on the screen as quickly as possibly when a presented stimulus matches that which is presented in the top right of the computer screen. Series of stimuli are presented in quick succession (rate of predefined and fixed digits per second) and participants must indicate at each match. For this test, the test accuracy (percentage of correct responses) and average reaction time (ms) can be recorded.

Cognitive Test 19: Choice Reaction Time Test

Test 19 will be performed using the screen at the base station. In this test participants are required to press the YES or NO button on the user interface as quickly as possible in response to the corresponding visual stimuli presented on the computer screen. A predefined number of presentations of the stimulus can be used in each test and can be presented at varying intervals. For this test, the accuracy of responses and average reaction time (ms) can be recorded.

Cognitive Test 20: Spatial Working Memory Test

Test 20 will be performed by showing a predefined set of white LEDs to be on for a specific period of time and the subject will be asked to memorize their location. The subject will be then shown another set of white LEDs to be on and they should press the Yes/No button on the hand controller 2400 to demonstrate if the new set of white LEDs similar to the first one or not. For this test, the sensitivity index (composite score of percentage of correctly identified stimuli and correctly rejected incorrect stimuli) and average reaction time (ms) can be recorded and used for later analysis.

Further examples of the system are now described according to several alternate embodiments:

Sensor, visual and audio packages referred below reference the following types of sensors and functionality:

Sensor Package A: Includes bodily fluid or breath-based sensors that detect chemical substances in a sample. Test results can be shown on a display or sent to a hub automatically for integration with system software.

Sensor Package B (e.g. for goggle integration): Sensors for measuring temperature, pulse rate, EEG, ECG, head movement (e.g. an accelerometer or gyroscope), and sweat and skin conductance (e.g. a galvanic skin response sensor).

Sensor Package C (e.g. for arm band integration): Sensors for measuring blood pressure, pulse rate, skin temperature, sweat and skin conductance, muscle tone (e.g. providing mechanical stimulation of tile muscle and measuring its frequency response), and EMG (electromyography).

Sensor Package D (e.g. for wrist band integration): Accelerometer, gyroscope, and magnetometer package to determine movements of the hand, and sensors for measuring skin temperature, pulse rate, sweat and skin conductance, ECG, muscle tone, mechanical stimulation of the muscle and measuring its frequency response, and EMG.

Sensor Package E (e.g. for comprehensive medical examination): Sensors for connection to a data collection system: electrocardiograph (ECG), pulse oximeter, blood pressure, wire 18-lead EEG, spirometer, thermometer, glucometer, blood analyzer, stethoscope, dermatoscope, otoscope, ophtalmoscope, endoscope, hand camera and ultrasound scanner.

Sensor Package F (e.g. for capturing user input): A remote controller with a few keys (e.g. left, right, up, down, OK, return, etc.), a joystick or gaming wheel with accelerate and/or brake pedals, a keyboard and/or mouse, a touchpad or touchscreen, and a speech recognition system that has one or more microphones to record a subject's voice and save the results.

Some drug impaired people will exhibit slurred or slow speech. It is important that impairment-detection physicians have access to the subject's recorded speech/voice when reviewing other test results to improve the diagnosis accuracy. In certain embodiments, this system has its own software to analyze the user input and responses. Therefore, the user's response to some auditory tests will be recorded and analyzed, and additionally, the user may have some type of interaction (such as skipping the current question, asking to repeat the question, etc.) with the testing with his/her voice commands if necessary. Sensor connections in this and other embodiments can be wired or have wireless communication with the main controller.

Sensor Package G: Intelligent/smart cameras that record RGB videos and/or depth and distance information. The underlying technology can be based on ultrasound, IR or other methods for measuring distance and speed. These cameras can be attached to the body of a cart or a frame. Alternatively, they can be placed on a tripod to record subjects' movements. All information will be sent to the system controller to be analyzed.

Sensor Package H: One or two lenses with fixed diopter values and/or electronically adjustable lenses where the diopter value of each lens can be controlled by an electrical command that is set by the software interface/microcontroller. The lenses should be placed somewhere between patient's eyes and LEDs. In one embodiment, there is one large lens that will be used by both eyes. Alternatively, one lens can be used for each eye (similar to some VR goggles). In both cases, a “reverse” lens can be attached to each camera to cancel out the effect of other lenses for proper eye recording. The lenses can also be capable of passing IR light.

Sensor Package I: One camera at the center or any other position inside the goggle, or two or more cameras to record from each eye separately or record eye movements from different angles. Cameras can be sensitive to IR light so they can record eye movements even when no visible light is available and eyes are only illuminated by IR light.

Sensor Package J: One or more accelerometers, gyroscopes, or inertial measurement units (IMU) to measure and record direction, orientation and position of the subject head or hand.

Visual Package K: A set of projectors and lights to project different patterns and images on a screen or on the ground.

Audio Package L: Internal or external speakers that are for example integrated with the goggles, computer device (Laptop/PC/Tablet/Phone, etc.) speakers, or external speakers that are connected with the main controller with wire or wireless communication technology. The audio system plays for example standard and consistent test instructions for the subject to follow for one or more parts of the test, voice instructions translated and played in different languages to make sure the subject fully understands the test instructions, follow different auditory signals to measure and quantify subject's brain response through other technologies such as EEG sensors, different tests such as simple math questions for the subject. The responses may be recorded with microphones explained in Package F and reviewed by an examiner or processed automatically by the intelligent speech recognition system.

Goggles and VR/AR goggles referred below utilize sensors, video and audio packages as described below, and have the following functionality according to certain embodiments:

A VR/AR Goggle System according to one embodiment: Includes a screen that shows some videos and test scenarios for impairment detection. Different scenarios can include for example: driving in different weather and road conditions (more like a driving simulator), cleaning windows of a tall building to test for phobia of heights, a police officer moving a pen from the right side of the screen to the left side, and different blocks in the subject's way and asking the subject to go around or jump over them. The system can have semantic features for scene creation. Test administrator can describe the desired test scenario verbally or written. The intelligent algorithms will use the test description to create the test visual and audio components automatically based on a set of predefined rules and algorithms. The system may have dynamic scenario construction features. Different scenarios mentioned above can be implemented with different complexity levels. Based on the users reaction, their movement, their balance, and their bio-signal readings, the scenarios can get more complex or less complex. This will help in quantifying the exact level of impairment of the user. Test instructions and/or training material can be administered. Alternatively, instructions may be played for the user through some external or internal speakers. Eye recording can be performed by Package 1 sensors that are integrated with the goggle headset. Biosignal sensors and communication can be implemented. Package B can be integrated with the goggle headset to measure bio signals as physiological feedback.

Package B can be implemented as some external sensors that communicate with goggle headset through the main controller. Communication can be wired or wireless. Head position and movements can be measured by integrated sensors of package J. User-Input can be implemented by Package F. The system can connect with wire/WIFI/Bluetooth to a router or laptop/PC/tablet to send and receive commands, test results and user inputs. Distance adjustment can be implemented by Package H to virtually increase/decrease the distance of projected light stimuli (can be a as simple as a dot moving from one side of the screen to the other side in the VR/AR screen) from the eyes of the subject. It helps the subjects with some sort of visual impairment to see light stimuli better and clearly to perform the test properly.

A goggle system according to one embodiment: Has an LED based light stimulus that may include: an LED array placed horizontally. In order to stimulate subjects' eyes at far end of each side of the goggle, the LED array can be extended by at least one set of shorter LED arrays at each end of the LED array in the middle. It may also include a vertical LED array, and an optical diffuser placed on the LED arrays to make movement of light stimuli from one LED to another one smoother. Alternatively, an LCD can be placed inside the goggle to show different light stimuli. Instructions may be played for the user through some external or internal speakers. Biosignal sensors and communication can be utilized. Package B can be integrated with the goggle headset to measure bio signals. Package B can be implemented as external sensors that communicate with goggle headset through the main controller. Communication can be wired/wireless. Head position and movements can be measured by integrated sensors of package J. Package F can be used to get user input. The system can connect with wire/WIFI/Bluetooth to a router or laptop/PC/tablet to send and receive commands, test results and user inputs. Package H can be used to virtually increase/decrease the distance of projected light stimuli (can be a as simple as a dot moving from one side of the screen to the other side in the VR/AR screen) from the eyes of the subject. It helps the subjects with some sort of visual impairment to see light stimuli better and clearly to perform the test properly.

Wristbands referred below utilize sensors packages as described below, and have the following functionality according to certain embodiments: Package D sensors are integrated. The band has rechargeable batteries that can be charged through an external power adaptor/USS or wirelessly. The band can have wired/wireless connections with goggle or VR/AR goggle embodiments.

Alternate system embodiments are now described:

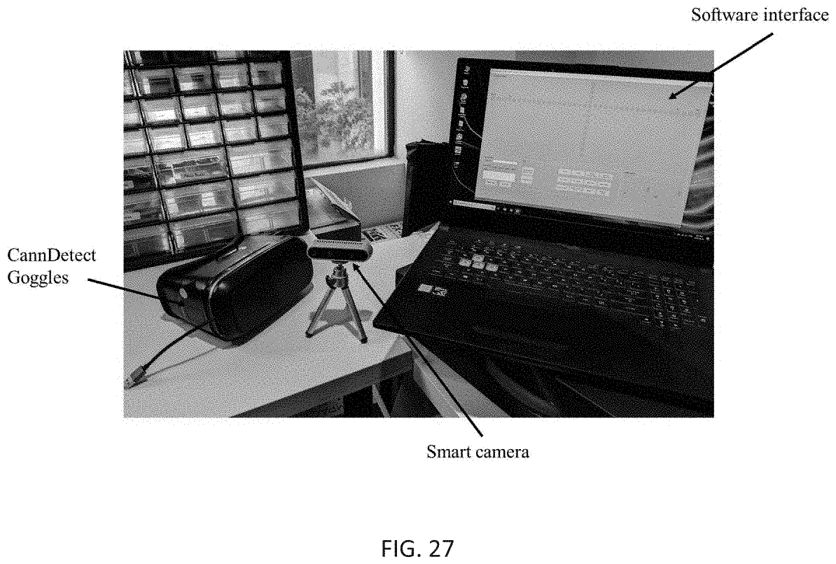

System components according to one embodiment include goggles, a wristband, sensors, a smart cameras, a system controller, a remote controller, a speaker for playing instructions, a projector system, an eye tracking system, a hub, a computer and software. Certain components of the system are shown in the setup of FIG. 27. In this form, a goggle or VR/AR goggle is used to perform and record neuro-cognitive tests and record head position/orientation measurements. Package L plays test instructions or auditory signals. Other bio-signals such as pulse rate and body temperature can be measured using integrated sensors in the goggles or through external sensors that are mentioned in Packages B, C and D. Data from sensors such as blood pressure and temperature can be entered and recorded in the software automatically or manually. In addition, some of the sensors can be integrated into a wristband system. Package G will be used to record balance and psychomotor tests. Package F will receive user input. Package K may receive commands from the main controller and project different patterns on the ground for the user based on the details of each tests (for example, projecting a straight line on the ground so that the subject can perform walk-and-turn test more accurately). The system may connect to Package A as well for chemical testing. All sensors' data and test results can be sent (wired or wireless communication) to a centralized hub, to be saved, reviewed and analyzed automatically (AI, machine learning, etc.) or manually by a human reviewer.

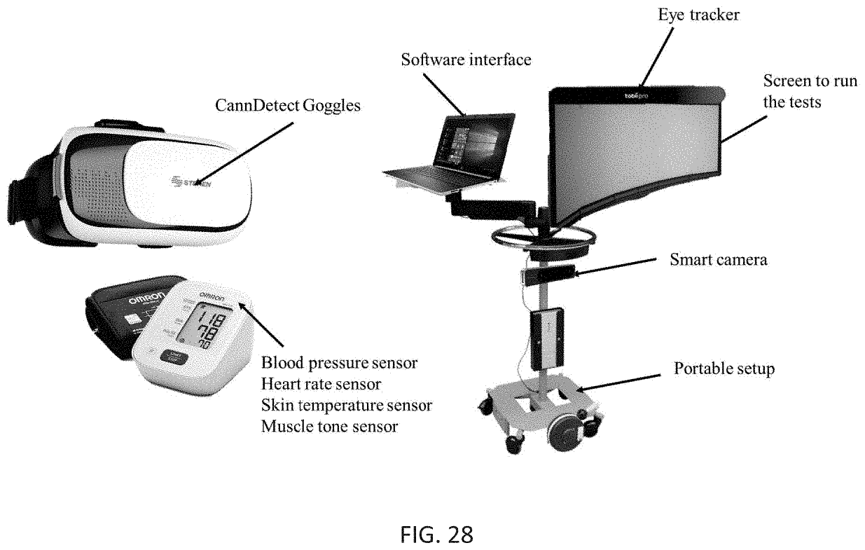

System components according to one embodiment include a stationary or portable cart, goggles, an eye tracker system, a screen, a wristband, sensors, a smart camera, speakers for playing instructions, a projector system, a system controller, a remote controller, a hub, a computer and software. Certain components are shown in the setup of FIG. 28. In this form, a goggle or VR/AR goggle is used to perform and record neuro-cognitive tests and record head position/orientation measurements.

Alternatively, a large screen may be used to show different patterns of light stimuli to the subject. In this embodiment, VR/AR goggles can still be used to perform neuro-cognitive tests as an external add-on. Package L will play test instructions or auditory signals. An eye tracker can be placed on the cart to record eye movements while subjects' eye are stimulated by the video that shown on the screen. Other bio-signals such as pulse rat e and body temperature can be measured using integrated sensors in the goggles or through external sensors that are mentioned in Packages B, C and D. External sensors are connected to the main hub and controller (wired or wireless).

Some of the sensors can be integrated into a wristband system. Package G will be used to record balance and psychomotor tests. Package F will receive user input. Package K may receive commands from the main controller and project different patterns on the ground for the user based on the details of each tests. An example of that can be projecting a straight line on the ground, so the subject can perform walk-and-turn test more accurately. The system may connect to Package A as well for chemical testing. All sensors' data and test results will be sent (wired or wireless communication) to a centralized hub, to be saved, reviewed and analyzed automatically or by a human reviewer.

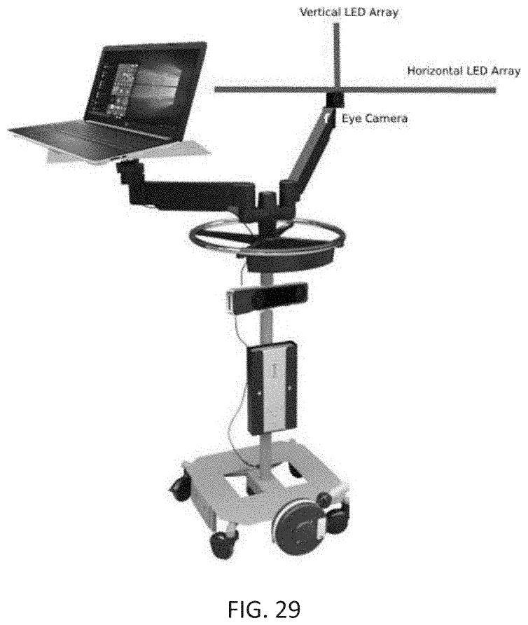

System components according to one embodiment include a stationary or portable cart, goggles, an eye tracker system, one or more LED arrays, a wristband, sensors, smart cameras, a speaker for playing instructions, a projector system, a system controller, a remote controller, a hub, a computer and software. Certain components are shown in the setup of FIG. 29. This system setup is similar to the embodiment of FIG. 28, however instead of using a screen, two LED arrays are used for horizontal and vertical light stimuli. Using long LED arrays are advantageous to simulating SFST eye tests accurately.

System components according to one embodiment include a box or container, and LED array or LCD screen, an eye tracker system, a wristband, sensors, smart cameras, a speaker for playing instructions, a projector system, a system controller, a remote controller, a hub, a computer and software. In this form, a screen (which might be as simple as an LCD array) or some LED arrays (horizontal and vertical) will be placed inside a big closed enclosure to show different patterns of light stimuli to the subject. In this case, the subject will watch the light stimuli through a gap designed in the frame of the box structure. However, a VR/AR goggle may be also used as an external add-on to perform and record neuro-cognitive tests; and record head position/orientation measurements. Package L will play test instructions or auditory signals. An eye tracker will be placed inside/on the box frame to record eye movements while subjects' eye are stimulated by the video that shown on the screen or by LED lights. Other bio-signals such as pulse rate and body temperature can be measured using integrated sensors inside the goggles or through external sensors that are mentioned in package B, C and D. External sensors are connected to the main hub and controller (wired or wireless). Some of the sensors can be integrated into a Wristband System. Moreover, some of the sensors such as temperature, pulse rate, galvanic skin response may be integrated with the box frame. Therefore, when the subject is watching the light stimuli and when some parts of his/her face are in touch with the frame, these sensors record the bio signals of interest. Package G will be used to record balance and psychomotor tests. Package F will receive user input. Package K may receive commands from the main controller and project different patterns on the ground for the user based on the details of each tests. An example of that can be projecting a straight line on the ground, so the subject can perform Walk and Turn test easier and more accurately. The system may connect to Package A as well for chemical testing. All sensors' data and test results will be sent (wired or wireless communication) to a centralized flub, to be saved, reviewed and analyzed automatically or by a human reviewer.

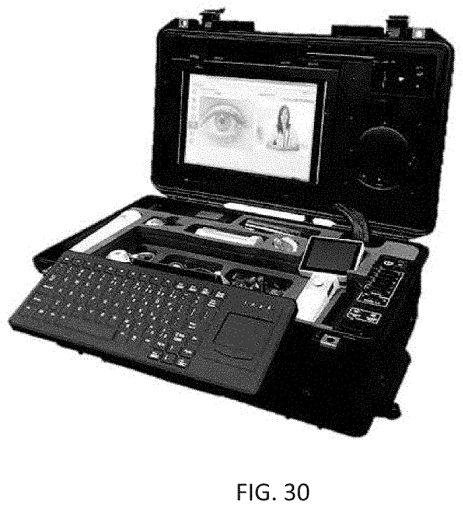

System components according to one embodiment include a box or briefcase containing various components which may include goggles, an eye tracker system, one or more LED arrays, a wristband, sensors, smart cameras, a speaker for playing instructions, a projector system, a system controller, a remote controller, a hub, a computer and software. Certain components are shown in the setup of FIG. 30. It's a portable package that may have its own screen, battery system and communication facilities. In this form, a goggle or VR/AR goggle can be used to perform and record eye and neuro-cognitive tests; and record head position/orientation measurements.

Alternatively, visual stimuli can be shown on the integrated screen. Package L will play test instructions or auditory signals. An eye tracker system is attached to the portable box that records eye response to the stimuli. Other bio-signals such as pulse rate and body temperature can be measured using integrated sensors in the goggles or through external sensors that are mentioned in package B. Some of the sensors can be integrated into a wristband system. An integrated implementation or external form of Package G can be used to record balance and psychomotor tests. Package F can receive user input. Package K may receive commands from the main controller and project different patterns on the ground for the user based on the details of each tests. An example of that can be projecting a straight line on the ground, so the subject can perform Walk and Turn test easier and more accurately. The system can connect to Package A as well for chemical testing. All sensors' data and test results can be sent (wired or wireless communication) to a centralized hub, to be saved, reviewed and analyzed automatically or by a human reviewer.

System components according to one embodiment include a tablet, mobile device, PC, laptop or cellphone, goggles, an eye tracker system, a wristband, sensors, smart cameras, a speaker for playing instructions, a projector system, a system controller, a hub, a computer and software. Embodiments include a portable package that can have its own screen, battery system and communication facilities. In this form, a goggle or VR/AR goggle can be used to perform and record eye and neuro-cognitive tests and record head position/orientation measurements. Alternatively, visual stimuli can be shown on the integrated screen. Package L can play test instructions or auditory signals. An eye tracker system is attached to the portable box that records eye response to the stimuli. Other bio-signals such as pulse rate and body temperature can be measured using integrated sensors in the goggles or through external sensors that are mentioned in package B. Some of the sensors can be integrated into a wristband system. An integrated implementation or external form of Package G will be used to record balance and psychomotor tests. Package F will receive user input. Package K can receive commands from the main controller and project different patterns on the ground for the user based on the details of each tests. An example of that can be projecting a straight line on the ground so the subject can perform a walk-and-turn test more easily and accurately. The system can connect to Package A as well for chemical testing. All sensors' data and test results will be sent (wired or wireless communication) to a centralized hub, to be saved, reviewed and analyzed automatically or by a human reviewer.

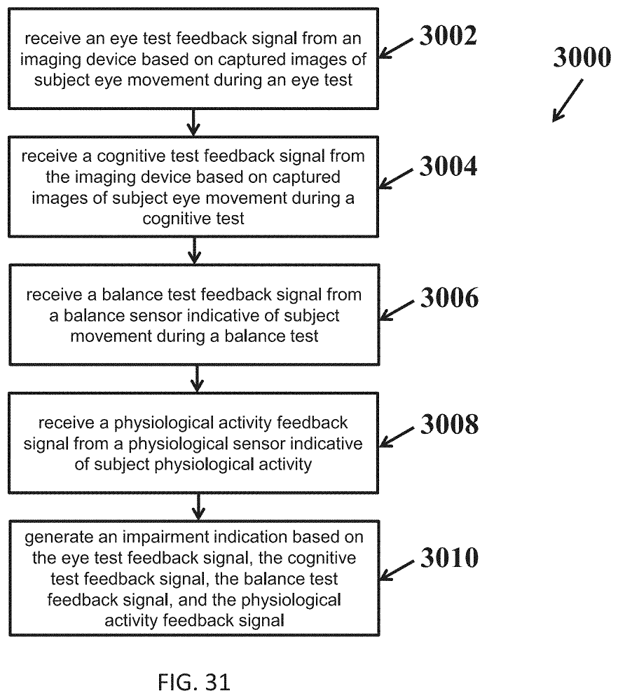

With reference now to FIG. 31, a method 3000 for screening impairment is shown according to one embodiment. The method includes the steps of receiving an eye test feedback signal from an imaging device based on captured images of subject eye movement during an eye test 3002, receiving a cognitive test feedback signal from the imaging device based on captured images of subject eye movement during a cognitive test 3004, receiving a balance test feedback signal from a balance sensor indicative of subject movement during a balance test 3006, receiving a physiological activity feedback signal from a physiological sensor indicative of subject physiological activity 3008, and generating an impairment indication based on the eye test feedback signal, the cognitive test feedback signal, the balance test feedback signal, and the physiological activity feedback signal 3010. In one embodiment, the method includes the step of generating an impairment determination signal based on the impairment indication. In one embodiment, the method includes the step of measuring the physiological activity indicated in the physiological activity feedback signal from a physiological sensor contacting the subject's skin.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

Example embodiments and criteria for determining impairment:

The “impaired” status can be identified based on one or more tests. For example, one or more tests needs to be “failed” such that the subject is considered to be “impaired”. Also, for many of the tests, a “failed” status can have a number associated with it (e.g. a 0 or 1 value or a percentage of a “fail” value). An integration of all percentage “fails” can determine the “impaired” or “not impaired” status.

Eye test: Eye tests are categorized into two groups of Dynamic Eye Tests and Static Eye Tests.

Dynamic Eye Tests: Each Dynamic Eye Test consists of a predetermined pattern of light movement and tracking the movement of the eye in response to the movement of the light. If the movement of the eye is different i11 terms of velocity or direction of movement is significantly different than the movement of light, the “fail” status will be identified. The difference should be greater than a predetermined threshold value to be considered significantly different.

Static Eye Tests: For these tests a static stimulation can be performed. For example, either all the visible lights will be turned off and the movement and size of the pupil will be tracked. In an alternative approach, a sudden change in the light intensity will be performed and the movement and size of the pupil will be tracked. In an alternative approach, specific lights will be turned on (e.g. the ones in the middle, or the ones in far left or far right), and the movement and size of the pupil will be tracked. The “fail” status can be assigned if the eye has jerky movements in Static Eye Tests (i.e. the eyes moves involuntarily in arbitrary positions, although the tests are static, and nothing is changing); or if the changes in the size of pupil (i.e. constriction or dilation) are significantly different than the normal eye reaction (i.e. either the amount of constriction or dilation is significantly different than normal eye, or the speed of constriction or dilation is significantly different than normal. eye).

Cognitive Tests: The cognitive tests can include multiple different tests. The main tests can be categorized into three groups of memory tests, reaction tests, and time conception tests.

Memory Tests: In the memory tests, the memory of the subject can be tested through a series of different tests. For example, a specific pattern of light can be shown to the subject for a specific period of time and they can be asked to memorize the pattern. Then a set of predetermined patterns can be shown, and the subject should say (either verbally or by clicking on the remote controller) “Yes” or “No” in response for each new pattern (e.g. “Yes” if the pattern is the same as the initial one and “No” if it is different). For this test the accuracy of the response (how many correct responses); the sensitivity of the response (meaning that how much the subject can remember, which means that can they remember patterns which are close to the original pattern as “Yes” or “No” or how much of difference they can distinguish); and the speed of responses are taken into consideration. The outcomes of these responses can determine if the subject “fails” the memory tests.

Reaction Tests: The reaction tests are designed to study the subject's reaction time. In an example of these tests, a visual stimulus can be presented using the visible lights and the subject is asked to click on the remote controller as soon as they see the light. The response time can be measured and recorded. The response time larger than a predetermined threshold value (in milliseconds) can identify the “fail” status for this test.

Time Conception Tests: The time conception tests are designed to study the subject's understanding of time. In an example of these tests, a visual stimulus can be presented for the subject. The subject will be asked to say (either verbally or by clicking a button on the remote controller) when they think a specific amount of time has passed. The difference between the subject's perception of passed time and the actual passed time is measured and recorded. A significant difference between the two times (difference larger than a predetermined threshold) can identify the “fail” status for t, his test.

Balance Tests: The balance tests can be done according to the Standard Field Sobriety Tests which include One Leg Stand (OLS) and Walk And Turn (WAT) tests. Each of these tests have specific number of clues to be identified. For example, WAT has 9 clues including no balance, starting too soon, stops walking, missed heel-to-toe, improper turn, etc. During these tests the movement of the subject will be recorded using one or more cameras. The recordings will then be evaluated either by an experienced reviewer or by automatic analysis of movement for the existence of the clues. Each clue has a “pass” or “fail” value associated with it (in a more general format, each clue will have a percentage of “pass” or “fail). “Failing” a certain number of clues for each test will constitute as “failing” that specific balance test.

Physiological Tests: The physiological tests include measuring multiple parameters from the subject's body. The tests can include, but are not limited to, body temperature measurement, blood pressure measurement, heart rate measurement, and muscle tone measurement. “Failing” each test means that the measured parameters significantly varies from the “normal” values. The “normal” values can either be determined with the subject baseline information (when the subject is “not impaired”) or by a predetermined value resulting from measuring the parameter on multiple subjects and averaging the values for a “normal” value. The significant deviation means a deviation larger than a predetermined threshold value.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A system for screening impairment of a subject comprising:
an imaging device and a display connected to a controller, the controller configured to send a first eye test signal to the imaging device and a second eye test signal to the display to initiate an eye test, and further configured to receive an eye test feedback signal based on captured images of subject eye movement during the eye test, and
the controller configured to send a first cognitive test signal to the imaging device and a second cognitive test signal to the display to initiate a cognitive test, and further configured to receive a cognitive test feedback signal based on captured images of subject eye movement during the cognitive test;
a balance sensor connected to the controller, the controller configured to receive a balance test feedback signal from the balance sensor indicative of subject movement during a balance test; and
a physiological sensor connected to the controller, the controller configured to receive a physiological activity feedback signal from the physiological sensor indicative of subject physiological activity;
wherein the controller is configured to generate an impairment indication based on the eye test feedback signal, the cognitive test feedback signal, the balance test feedback signal, and the physiological activity feedback signal.

2. The system of claim 1, wherein the controller is configured to send an impairment determination signal to the display based on the impairment indication.

3. The system of claim 1, wherein the imaging device comprises a first camera and second camera.

4. The system of claim 1, wherein the display comprises a plurality of light elements or a display screen.

5. The system of claim 4, wherein the plurality of fight elements is a plurality of LED elements or a linear array of light elements of a plurality of linear arrays of light elements.

6. The system of claim 5 further comprising:
an optical diffuser configured to cover the plurality of light elements.

7. The system of claim 5, wherein a first array of the plurality of linear arrays of light elements is disposed horizontally and/or a second array of the plurality of linear arrays of light elements is disposed vertically.

8. The system of claim 5, wherein the plurality of light elements includes a plurality of linear arrays of light elements disposed parallel to each other and/or disposed perpendicular to each other.

9. Goggles comprising the system of claim 1, wherein the display is configured within a viewing cavity.

10. The goggles of claim 9 further comprising:
an administrator display configured on an external surface of the goggles;
an administrator display configured out of the subject's view during testing.

11. The system of claim 1 further comprising: an imaging illumination element.

12. The system of claim 11, wherein the illumination element is an infrared light element.

13. The system of claim 1, wherein the balance sensor is an accelerometer, gyroscope, magnetometer, shoe or insole force sensor, or wearable activity monitoring sensor; or wherein the imaging device functions as the balance sensor.

14. The system of claim 1, wherein the physiological sensor is a heart rate sensor, a blood pressure sensor, a body tremor sensor, an oral moisture sensor, an electrodermal activity monitor, a body temperature sensor, sweat and skin conductance sensor, a muscle tone sensor, a frequency response sensor, an electromyography sensor, a glucometer, a blood analyzer, a stethoscope, a dermatoscope, an otoscope, an ophthalmoscope, an endoscope and an ultrasound scanner.

15. The system of claim 1, wherein the eye test comprises at least one of a resting nystagmus eye test, a horizontal gaze nystagmus eye test, a vertical gaze nystagmus eye test, a lack of smooth pursuit eye test, an equal pupil eye test, a nystagmus at maximum deviation eye test, a nystagmus prior to 45 degrees eye test, a non-convergence eye test, a pupil rebound dilation test, a Hippus test, a red-eye (bloodshot) test, a watery eye test, and an eyelid twitching test.

16. The system of claim 1, wherein the controller is configured to generate the impairment determination signal based on at least one of image analysis, data analysis, data visualization, and data integration.

17. The system of claim 1 further comprising:
a hand controller configured to measure a reaction time to a light signal or to an auditory signal.

18. A method for screening impairment of a subject comprising:
receiving an eye test feedback signal from an imaging device based on captured images of subject eye movement during an eye test;
receiving a cognitive test feedback signal from the imaging device based on captured images of subject eye movement during a cognitive test;
receiving a balance test feedback signal from a balance sensor indicative of subject movement during a balance test;
receiving a physiological activity feedback signal from a physiological sensor indicative of subject physiological activity; and
generating an impairment indication based on the eye test feedback signal, the cognitive test feedback signal, the balance test feedback signal, and the physiological activity feedback signal.

19. A system for screening impairment of a subject comprising:
an imaging device and a display connected to a controller, the controller configured to send a first eye test signal to the imaging device and a second eye test signal to the display to initiate an eye test, and further configured to receive an eye test feedback signal based on captured images of subject eye movement during the eye test, and
the controller configured to send a first cognitive test signal to the imaging device and a second cognitive test signal to the display to initiate a cognitive test, and further configured to receive a cognitive test feedback signal based on captured images of subject eye movement during the cognitive test;
wherein the controller is configured to generate an impairment indication based on the eye test feedback signal and the cognitive test feedback signal.

20. The system of claim 19 further comprising:
a balance sensor connected to the controller, the controller configured to receive a balance test feedback signal from the balance sensor indicative of subject movement during a balance test;
wherein the controller is configured to generate the impairment indication based on the balance test feedback signal;
a physiological sensor connected to the controller, the controller configured to receive a physiological activity feedback signal from the physiological sensor indicative of subject physiological activity;
wherein the controller is configured to generate the impairment indication based on the physiological activity feedback signal.
